(19) United States
(12) Patent Application Publication
(10) Pub. No.: US 2007/0037199 A1
(43) Pub. Date: Feb. 15, 2007

(54) Individual discriminating method, as well as array, apparatus and system for individual discriminating test

(76) Inventors: Masayoshi Takahashi, Kawasaki-shi (JP); Koji Hashimoto, Atsugi-shi (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(21) Appl. No.: 11/514,894

(22) Filed: Sep. 5, 2006

Related U.S. Application Data
(63) Continuation of application No. PCT/JP06/13198, filed on Jun. 27, 2006.

(30) Foreign Application Priority Data
Jun. 28, 2005 (JP) 2005-188452

(51) Int. Cl. C12Q 1/68; G06F 19/00; C12M 3/00

(52) U.S. Cl. 435/6; 435/287.2; 702/20

(57) ABSTRACT

The invention provides a method of individual discriminating. The method includes selecting and using single nucleotide polymorphism satisfying any one of the conditions defined by this invention.

### CROSS-REFERENCE TO RELATED APPLICATIONS

This is a Continuation Application of PCT Application No. PCT/JP2006/313198, filed Jun. 27, 2006, which was published under PCT Article 21(2) in English.

This application is based upon and claims the benefit of priority from prior Japanese Patent Application No. 2005-188452, filed Jun. 28, 2005, the entire contents of which are incorporated herein by reference.

### BACKGROUND OF THE INVENTION

1. Field of the Invention

The present invention relates to a method of discriminating individual using DNA sequence information, and further, relates to an array, an apparatus and a system for an individual discriminating test.

2. Description of the Related Art

By almost complete decoding of the nucleotide sequence of the human genome, a sequence having a difference between individuals and between races, and the frequency thereof has been made clear. The difference between individuals is utilized in studies for elucidating the relationship between the effect and the side effects of a drug in the medical field.

The difference in a genome nucleotide sequence is also utilized in individual identification in criminal investigation. Currently, the main method used in individual identification is a method based on a difference in a repetition time of a repeat sequence on a DNA sequence, a representative of which is Short Tandem Repeat (STR) and Variable Number of Tandem Repeat (VNTR). For example, a method which has been adopted by the police since 2003 is a method of amplifying a region of nine places of STR and one place of VNTR by a polymerase chain reaction (PCR), subjecting the amplification product to a capillary electrophoresis method, and determining the repetition number from mobility thereof.

However, in a sequence such as STR, VNTR and the like, a constant sequence of a few nucleotide units appears repeatedly, and regarding the aforementioned ten places, a full length of the repeat sequence is about 30 to 600 bp. In order to measure this repetition time, it is required that an objective region is retained in a sample to be tested without being cleaved. However, it is easily considered that a body liquid and a blood trace left at the scene of sample collecting for criminal investigation, or a sample left at the scene of a terrible disaster such as fire, explosion, accident and the like has been deteriorated. In such the case, there is a possibility that a DNA sequence has been fragmented, and an entire objective region has not been retained. Like this, in a discriminating method with a repeat sequence requiring a relatively long sequence, there is a problem that measurement is difficult in some cases, and therefore, an as small as possible DNA region necessary for a test is preferable.

Then, a method using single nucleotide polymorphism for individual identification which has been elucidated in recent years is contemplated. For example, JP-A 2004-239766(KOKAI) describes that it is possible to use single nucleotide polymorphism in a DNA probe of a microarray for discriminating an individual. However, its specific method is not disclosed. In addition, Lee H Y, et. al. (Selection of twenty-four highly informative SNP markers for human identification and paternity analysis in Koreans.; Forensic Aci Int. 2005 Mar. 10; 148(2-3):107-12.) discloses 24 nucleotide polymorphisms which can be used in identification of an individual and parentage test in the Korean population.

However, there are a huge number of single nucleotide polymorphisms which are found now, and it is not practical to test all of them. However, as to what single nucleotide polymorphism should be tested, there is no powerful determination criteria now.

An object of the present invention is to provide a method which allows for simple and rapid individual discrimination by selecting single nucleotide polymorphism which is advantageous for individual discrimination. Also the invention provides an array, a test apparatus and a system for use in an individual discriminating test.

### BRIEF SUMMARY OF THE INVENTION

According to the present invention, there is provided an individual discriminating method for determining consistency between a nucleotide sequence possessed by an individual and a nucleotide sequence possessed by a sample, comprising a step of selecting a plurality of single nucleotide polymorphism from a group of single nucleotide polymorphisms present in a population to which a subject individual to be discriminated belongs, and a step of determining genotypes in the selected plurality of single nucleotide polymorphisms in the nucleotide sequence possessed by a subject individual and the nucleotide sequence derived from a sample, and discriminating an individual by comparing the genotypes, wherein in the selecting step, single nucleotide polymorphism satisfying any one of the following conditions (i) to (iii) is selected:

(i) in case of a 2-nucleotide substitution type single nucleotide polymorphism, assuming that respective allele frequencies of two possible nucleotides are X and Y, X+Y=1, and Y≦X;

0.5≦X≦0.7,

(ii) in case of a 3-nucleotide substitution type single nucleotide polymorphism, assuming that respective allele frequencies of three possible nucleotides are X, Y and Z, X+Y+Z=1, and Z≦Y≦X;

1/3≦X and (1-X)/2≦Y and X+Y<1, and

(a) Y≦1/2·X and X<2/3, or (b) 1/2·X<Y and XY<2/9, and

(iii) in case of a 4-nucleotide substitution type single nucleotide polymorphism, assuming that respective allele frequencies of four possible nucleotides are X, Y, Z and W, X+Y+Z+W=1, and W≦Z≦Y≦X;

1/4≦X and (1-X)/3≦Y and X+Y<1, and

(a) Y≦1/2·X and X<2/3, or (b) 1/2·X<Y and XY<2/9.

Herein, the X is preferably 0.55≦X<0.7, more preferably 0.6≦X<0.7, and further preferably 0.65≦X≦0.68.

Also, according to another aspect of the present invention, there is provided an array in which nucleic acid probes are fixed to a substrate, for use in an individual discriminating test for determining consistency between a nucleotide sequence possessed by an individual and a nucleotide sequence possessed by a sample, characterized in that the nucleic acid probes have a sequence complimentary to a target sequence containing single nucleotide polymorphism, and the single nucleotide polymorphism is selected from a group of single nucleotide polymorphisms present in a population to which a subject individual to be discriminated belongs, and satisfies any one of the following conditions (i) to (iii):

(i) in case of a 2-nucleotide substitution type single nucleotide polymorphism, assuming that respective allele frequencies of two possible nucleotides are X and Y, X+Y=1, and Y≦X;

0.5≦X≦0.7,

(ii) in case of a 3-nucleotide substitution type single nucleotide polymorphism, assuming that respective allele frequencies of three possible nucleotides are X, Y and Z, X+Y+Z=1, and Z≦Y≦X;

1/3≦X and (1-X)/2≦Y and X+Y<1, and

(a) Y≦1/2·X and X<2/3, or (b) 1/2·X<Y and XY<2/9, and

(iii) in case of a 4-nucleotide substitution type single nucleotide polymorphism, assuming that respective allele frequencies of four possible nucleotides are X, Y, Z and W, X+Y+Z+W=1, and W≦Z≦Y≦X;

1/4≦X and (1-X)/3≦Y and X+Y<1, and

(a) Y≦1/2·X and X<2/3, or (b) 1/2·X<Y and XY<2/9.

Herein, the X is preferably 0.55≦X<0.7, more preferably 0.6≦X<0.7, further preferably 0.65≦X<0.68.

### BRIEF DESCRIPTION OF THE SEVERAL VIEWS OF THE DRAWING

FIG. 1A is a view showing a relationship between an allele frequency of a 2-nucleotide substitution type SNP and a genotype frequency.

FIG. 1B is Extraction of a maximum genotype frequency and a minimum genotype frequency from FIG. 1A.

FIG. 2 is a view showing a relationship between an allele frequency of a 3-nucleotide substitution type SNP and a genotype frequency.

FIG. 3 is a view showing a relationship between an allele frequency of a 4-nucleotide substitution type SNP and a genotype frequency.

FIG. 4 is a view showing an example of calculating an existence probability.

FIG. 5 is a conceptional view showing a whole construction of an individual discriminating test system.

FIG. 6A is a view showing details of a construction of a chip cartridge relating to a first embodiment.

FIG. 6B shows a view of FIG. 6A seen in a B-B direction.

FIG. 6C shows a partial perspective cross-sectional view of FIG. 6A seen in a C-C direction.

FIG. 6D shows a view of FIG. 6A seen in a D-D direction from a back.

FIG. 7 is a view showing a support and a lid on a chip cartridge before fixation with an upper lid fixing screw.

FIG. 8 is a view showing a detailed construction of a printed board on which an individual discriminating chip is packaged.

FIG. 9A is a view showing a cell and a drug supplying system communicating with the cell relating to a first embodiment.

FIG. 9B is a top view of FIG. 9A.

FIG. 9C is a view showing a part of FIG. 9A.

FIG. 10A is a view showing a more detailed construction of each constitutional element in vicinity of a cell.

FIG. 10B is a view showing appearance in which a chip cartridge upper lid is fixed to a chip.

FIG. 11 is a top view of a cell relating to a first embodiment.

FIG. 12 is a top view of a variation example of a cell relating to a first embodiment.

FIG. 13A is a cross-sectional view of a process for manufacturing an individual discriminating chip and a printed board.

FIG. 13B is a view follows FIG. 13A.

FIG. 13C is a view follows FIG. 13B.

FIG. 13D is a view follows FIG. 13C.

FIG. 14 is a top view of an individual discriminating chip.

FIG. 15 is a view showing one example of the flow system relating to a first embodiment.

FIG. 16 is a view showing a flowchart of a flowing step for an individual discriminating test using a flow system.

FIG. 17 is a view showing the measuring system relating to a first embodiment.

FIG. 18 is a view showing the previous potentiostat.

FIG. 19 is a view showing one example of a procedure for analyzing measurement data.

FIG. 20 is a view showing a flowchart of type determination filtering treatment.

FIG. 21 is a view showing one example of type determination treatment.

FIG. 22 is a sequence view of an automatic analyzing procedure for individual discrimination using an individual discrimination testing apparatus.

FIG. 23A is a graph showing measurement results of an example of genotype detection.

FIG. 23B is a graph showing measurement results of an example of genotype detection.

### DETAILED DESCRIPTION OF THE INVENTION

According to one aspect of the present invention, an individual discriminating method is provided. In the present specification, individual discrimination means determination of consistency between a nucleotide sequence possessed by an individual and a nucleotide sequence possessed by a sample. For example, the method can be used for specifying whether an article left such as a remaining blood trace and a hair is of a suspect or a victim or not in a criminal investigation. Therefore, a nucleotide sequence possessed by a sample may be a sequence of a nucleic acid contained in these articles left, being not limited thereto.

As used herein, the term “nucleic acid” is a term comprehensively indicating a nucleic acid and a nucleic acid analogue such as a ribonucleic acid (RNA), a deoxyribonucleic acid (DNA), a peptide nucleic acid (PNA), a methylphosphonate nucleic acid, a S-oligo, a cDNA and a cRNA, as well as any oligonucleotide and polynucleotide. Such the nucleic acid may be naturally occurring, or may be artificially synthesized.

Herein, a nucleotide sequence is preferably a genome DNA sequence, or may be a fragmental sequence which does not ensure a whole genome. In addition, individual discrimination of the present invention can be also used in person identification for confirming a person himself, parentage test or the like, being not limited thereto.

In the method of the present invention, single nucleotide polymorpholism (SNP) is used for individual discrimination, and particularly, SNP which is advantageous in individual discrimination is selected and used. This selection of SNP is performed by referring to its allele frequency.

Herein, SNP refers to a difference in a genome DNA sequence by one nucleotide, and this is recognized in a specified population usually at a frequency of 1% or more. In almost cases, in one place SNP, substitution occurs between two nucleotides, and for example, in an individual, A (adenine) is taken, and in another individual, G (guanine) is taken. Such the SNP is called 2-nucleotide substitution type, and in this case, there are three ways of genotypes in one place SNP. In the above example, there are A/A homo, G/G homo, and A/G hetero.

However, rarely, a 3-nucleotide substitution type or 4-nucleotide substitution type SNP is also present. In these SNPs, there are six or ten ways of genotypes, so only at one SNP site, there can be a number of genotypes.

An appearance frequency of these genotypes can be obtained from an allele frequency of each nucleotide. An allele frequency refers to each ratio of a nucleotide which can be taken at a certain SNP in a population. That is, when certain SNP in a population takes any nucleotide of A and T, the case of A in the population is a ratio of 70%, and the case of T is a ratio of 30%, an allele frequency X of A is expressed to be 0.7, and an allele frequency Y of T is expressed to be 0.3. Herein, since X+Y=1, and this is 2-nucleotide substitution, 0<X, and 0<Y.

This allele frequency is determined by testing a considerable number of individual genotypes contained in a population, and obtaining its distribution. Currently, allele frequencies in various populations are published by a plurality of databases, and populations according to various classifications such as a race and people have been investigated, respectively. As database listing a position and a frequency of SNP, for example, there are HAPMAP, NCBI Entrez SNP, JSNP, TSC and the like.

In the present specification, an individual group classified by these classifications, for example, race, people, nation, residence region, sex, age and the like is referred to as population. The number of individuals investigated for determining an allele frequency is suitable, and if highly reliable, an allele frequency published by any database may be used, or investigation of an allele frequency may be performed independently.

As described above, a genotype frequency is obtained from an allele frequency. In the case of the above SNP, as the genotype, there are AA homo, AT hetero and TT homo, and a frequency of each genotype is obtained from (X+Y)²=XX+2XY+YY. That is, AA:AT:TT=0.49:0.42:0.09.

In the method of the present invention, proper SNP is selected depending on these allele frequency and genotype frequency. In a nucleotide sequence of an individual and a nucleotide sequence of a sample, genotypes in the selected SNPs are determined, and they are compared. If both genotypes are entirely consistent, it can be determined that the sample is derived from that individual.

In addition, in the present specification, an individual may be any entity such as an animal and a plant in addition to a human, as far as the entity can be discriminated by a genome DNA sequence. A preferable subject is a human, and additionally, a livestock and a pet, or a cultivating plant or a wild plant may be included.

Meanwhile, it is said that around ten millions of SNPs are present in a human genome DNA. Although when as many as possible, SNPs are tested, a test precision is apparently elevated, in view of simplicity, rapidness and economy, it is apparent that a small number of SNPs to be tested is better.

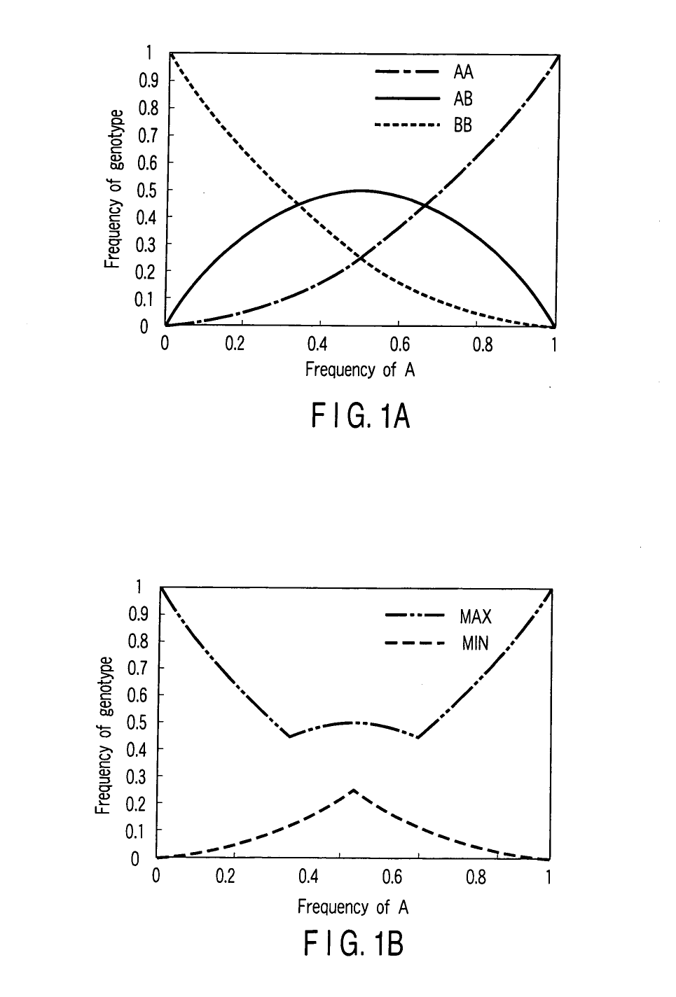

Then, the present inventors found that SNP which is advantageous in individual discrimination can be selected according to the following conditions, and this allows for a minimum number of SNPs necessary for individual discrimination. Conditions (i) to (iii) for selecting SNP will be successively explained below. (i) First, a method of selecting SNP in the case of a 2-nucleotide substitution type will be explained. Letting nucleotides being substituted to be A and B, a relationship between an allele frequency and a genotype frequency is shown in FIG. 1A. In FIG. 1A, as a frequency of A is increased, a frequency of a genotype AA is increased. Conversely, as a frequency of A is increased, a frequency of B is decreased, and a frequency of a genotype BB is also decreased. When a frequency of A is 0.5, that is, frequencies A and B are the same, a frequency of a genotype AB becomes maximum.

Extraction of a maximum genotype frequency and a minimum genotype frequency in this FIG. 1A is FIG. 1B. As shown in this figure, in a 2-nucleotide substitution type SNP, a genotype of a highest genotype frequency (MAX) takes a lowest genotype frequency of 4/9 at a frequency of A of 0.66, and is not lowered therefrom. In addition, a genotype of a lowest genotype frequency (MIN) becomes a maximum genotype frequency at a frequency of A of 0.5, and is decreased around it.

Herein, what a genotype frequency possessed by SNP is desirable in individual discrimination will be considered. In certain SNP, it is desirable that a frequency of a genotype having a maximum genotype frequency is as low as possible. This is because when a maximum genotype frequency is high, this leads to many individuals having the genotype in the population, and the ability to discriminate an individual of the SNP is reduced. Therefore, in order to ensure lowest limit of discrimination ability, it is preferable that a frequency of a genotype having a maximum genotype frequency is 0.5 or less.

In addition, it is desirable that a frequency of a genotype having a minimum genotype frequency is as low as possible. When this genotype frequency is low, it can be said that the genotype is rare, and it can be said that the SNP has extremely high discriminating ability, and is useful SNP.

Herein, respective allele frequencies of nucleotides A and B are expressed by X and Y. It is provided that X+Y=1, and Y≦X. In addition, from definition, 0<X and 0<Y, and from these conditions, 0.5≦X. Thereupon, in FIG. 1B, X in which a maximum genotype frequency (max) is not more than 0.5, leads to 0.5≦X≦0.7. Hence, SNP in which an allele frequency is 0.5≦X≦0.7 is suitably used.

Herein, as described above, in order to decrease a maximum gene frequency, it is desirable that a value of X approaches 0.66. However, in order to decrease a minimum genotype frequency, it is preferable that the value of X is greater. In view of these conditions, a further preferable range is a range of 0.55≦X<0.7, more preferably a range of 0.6≦X<0.7, most preferably a range of 0.65≦X≦0.68.

SNP having X (allele frequency) of the aforementioned range has better balance in discriminating ability, and such the SNP can be preferably used in the method of the present invention.

(ii) Then, a method of selecting SNP in the case of a 3-nucleotide substitution type will be explained. There are six ways of genotypes in the 3-nucleotide substitution type SNP as described above. Therefore, discriminating ability by one SNP is improved, and this is advantageous for use in discrimination of an individual.

Further, in the 2-nucleotide substitution type, a minimum genotype frequency which can be taken by a maximum genotype frequency is 4/9. Therefore, in the 3-nucleotide substitution type, it is possible to select SNP more effectively in individual discrimination by selecting SNP having a smaller genotype frequency.

Herein, it is provided that nucleotides being substituted are A, B and C, and allele frequencies are X, Y and Z, respectively. Herein, X+Y+Z=1, and Z≦Y≦X. Thereupon, 1/3≦X, (1-X)/2≦Y, and X+Y<1.

Meanwhile, genotypes in the 3-nucleotide substitution type are X², Y², Z², 2XY, 2YZ, and 2ZX. Among them, genotypes in which a genotype frequency can be maximal are X² or 2XY. (a) In the case of 2XY≦X², that is, Y≦1/2·X, a maximum genotype is X². Herein, as described above, since a genotype frequency smaller than 4/9 is desirable, X²<4/9. Therefore, a desirable allele frequency is Y≦1/2·X and X<2/3, and such the SNP is selected.

Or, (b) in the case of 2XY>X², that is, 1/2 X<Y, a maximum genotype is 2XY. Herein, as described above, since a genotype frequency smaller than 4/9 is desirable, 2XY<4/9. Therefore, a desirable allele frequency is 1/2·X<Y and XY<2/9, and such the SNP is selected.

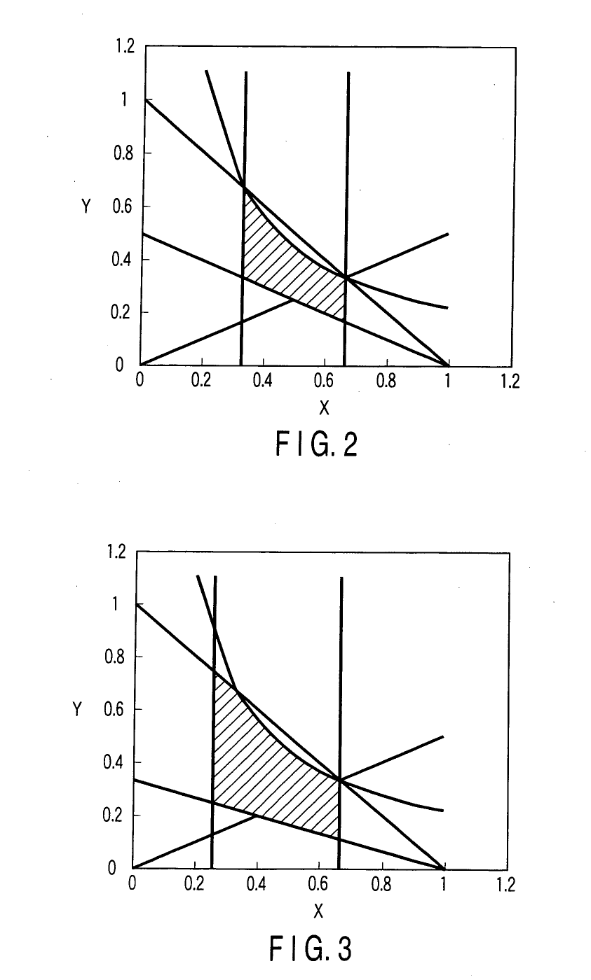

A range of X and Y in conformity with the above conditions (a) and (b) is shown as a region of an oblique line in FIG. 2. The 3-nucleotide substitution type SNP having this range of an allele frequency is SNP which can contribute to individual discrimination more than the 2-nucleotide substitution type SNP, and can be said to be SNP having high efficiency.

(iii) Then, a method of selecting SNP in the case of a 4-nucleotide substitution type will be explained. There are ten ways of genotypes in the 4-nucleotide substitution type SNP as described above. Therefore, discriminating ability by one SNP is greatest, and this is advantageous for use in individual discrimination. In the 4-nucleotide substitution type SNP like the 3-nucleotide substitution type SNP, by selecting SNP having a genotype frequency in which a maximum genotype frequency is smaller than 4/9, it is possible to select SNP more effectively in individual discrimination.

Herein, it is provided that nucleotides being substituted are A, B, C and D, and allele frequencies are X, Y, Z and W, respectively. Herein, X+Y+Z+W=1, and W≦Z≦Y≦X. Thereupon, 1/4≦X, (1-X)/3≦Y, and X+Y<1.

Genotypes in the 4-nucleotide substitution type in which a genotype frequency can be maximum are X² and 2XY.

(a) In the case of 2XY≦X², that is, Y≦1/2·X, a maximum genotype is X². Herein, as described above, since a genotype frequency smaller than 4/9 is desirable, X²<4/9. Therefore, a desirable allele frequency is Y≦1/2·X and X<2/3, and such the SNP is selected.

Or, (b) in the case of 2XY>X², that is, 1/2·X<Y, a maximum genotype is 2XY. Herein, as described above, since a genotype frequency smaller than 4/9 is desirable, 2XY<4/9. Therefore, an desirable allele frequency is 1/2·X<Y and XY<2/9, and such the SNP is selected.

A range of X and Y in conformity with the above (a) and (b) is shown as an oblique line region in FIG. 3. The 4-nucleotide substitution type SNP having this range of an allele frequency is SNP which can contribute to individual discrimination more than the 2-nucleotide substitution type SNP, and can be said to be SNP having high efficiency.

SNP shown in the above (ii) and (iii) can be searched, for example, from SNP registered in SNP database of National Center for Biological Information (NCBI).

It is desirable that SNP selected so that the conditions (i) to (iii) are satisfied is selected from different chromosomes in a genome. When using SNPs present on the same chromosome, it is desirable to use SNPs being clearly not linked to each other, or SNPs having a low probability thereof.

In addition, it is desirable that a combination of a plurality of SNPs selected according to the above conditions is selected so that, in a genotype frequency calculated from an allele frequency, each of selected i single nucleotide polymorphisms, letting a highest genotype frequency in a n^{th} single nucleotide polymorphism to be (Max)ₙ and a lowest genotype frequency to be (Min)ₙ, 1/π(Max)ᵢ and 1/π(Min)ᵢ become suitable values, respectively.

For example, letting the number of individuals constituting the population to be U, SNP can be also selected so that each of 1/π(Max)ᵢ and 1/π(Min)ᵢ is in an appropriate range when expressed as a ratio to U. 1/π(Max)ᵢ represents that, in a combination of selected SNPs, an individual having a combination of a highest probability is present at a ratio of one person per 1/π(Max)ᵢ persons. When a value of 1/π(Max)ᵢ is low, many individuals having the combination of SNPs are present in a population, and it can be said that the combination of SNPs has low individual discriminating ability.

In addition, 1/π(Min)ᵢ represents that, in a combination of selected SNPs, an individual having a combination of a lowest probability is present at a ratio of one person per 1/π(Min)ᵢ persons. When a value of 1/π(Min)ᵢ is low, an individual having the combination of SNPs is rarely present, and it can be said that the combination is a combination of SNPs having high individual discriminating ability.

A number i of SNPs selected for use in individual discrimination in the invention may be appropriately determined depending on necessary discriminating ability, and may be determined in view of simplicity, rapidness, economy, and reliability of determination.

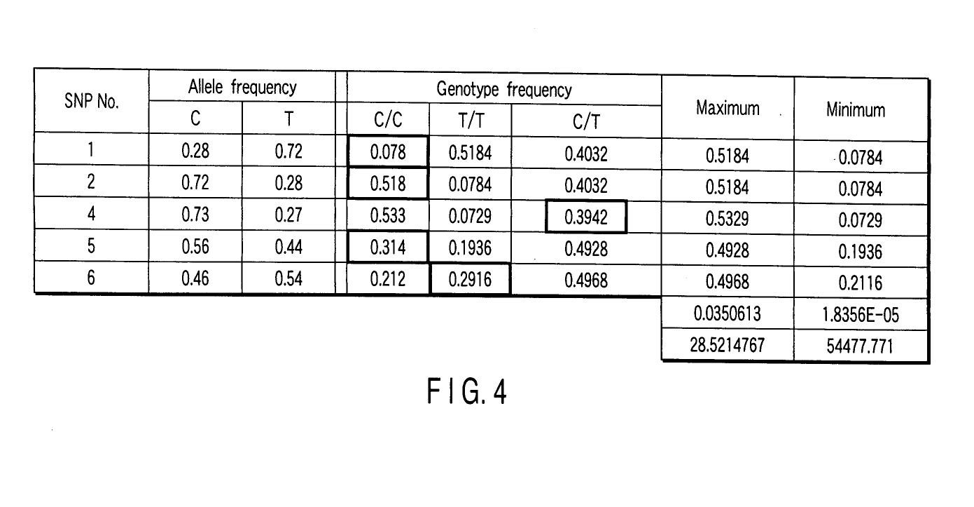

In an individual discriminating method using SNPs selected according to the aforementioned conditions, further, by calculating a frequency of a genotype possessed by a subject individual from an allele frequency, multiplying a genotype frequency regarding all selected SNPs, and taking a reciprocal, a probability that a combination of genotypes possessed by a subject individual is present in a population can be calculated. To explain using FIG. 4, when genotypes of a subject individual are 1:CC, 2:CC, 3:CT, 4:CC, and 5:TT, multiplication of respective genotype frequencies gives 0.078×0.518×0.3942×0.314×0.2916=0.001458, and a reciprocal thereof is 685.7. Therefore, a genotype of this sample is a genotype which is presented one person per 686 persons.

For example, in the case of a genotype possessed by one person per ten persons in a population of 100 millions, even when genotypes of a subject individual and a sample are consistent, reliability that they are the same is decreased. Conversely, in the case of a genotype, when a genotype possessed by a subject individual is a genotype which is rarely present, reliability of a determination of is increased, therefore, it is also important upon individual discrimination to determine a probability of existence in a population, and make clear reliability of determination of whether a nucleotide sequence possessed by an individual and a nucleotide sequence of a sample are consistent or not. By calculating an existence probability by this step, reliability of the result of individual discrimination can be determined.

The individual discriminating method of the present invention which has been described in detail above is summarized. First, a plurality of SNPs satisfying any of the above (i) to (iii) are selected from a group of SNPs present in a population to which a subject individual to be discriminated belongs. Herein, genotypes in selected plurality of single nucleotide polymorphisms are determined, respectively, in a nucleotide sequence possessed by a subject individual and a nucleotide sequence derived from a sample. Then, determined genotypes of both of them are compared, and it is determined whether a nucleotide sequence of a subject individual and a nucleotide sequence of a sample are consistent or not based on the comparison results.

Herein, a method of determining genotypes in nucleotide sequences of a subject individual and a sample may be performed by the well-known method, for example, an appropriate method such as by a method of amplifying the SNP site by a polymerase amplification reaction, and investigating a genotype, and a method of analyzing a DNA sequence.

By using SNPs selected according to the present invention, an individual can be discriminated with a minimum number of SNPs within required reliability, a test can be performed simply and rapidly, and the cost can be suppressed.

Then, according to another aspect of the present invention, an array for use in an individual discriminating test is provided. The array in the present invention is such that nucleic acid probes having a sequence complementary with a target nucleic acid is fixed to a substrate. Herein, a target nucleic acid means a nucleic acid strand having a target sequence containing SNP selected from a group of SNPs present in a population to which a subject individual to be discriminated belongs so as to satisfy any one of the above conditions (i) to (iii). The nucleic acid probes fixed to a substrate can hybridize with a target nucleic acid under the appropriate condition.

As used herein, the terms “complementary”, “complementarity” and “complementation” are enough to be complementary in a range of 50% to 100%, preferably refer to 100% complementary.

The nucleic acid probes fixed to a substrate may be a single nucleic acid probe, or a plurality different nucleic acid probes. That is, nucleic acid probes having sequences which are complementary with target nucleic acids having target sequences containing different SNPs, respectively, may be fixed.

In addition, for example, in SNP of A and T 2-nucleotide substitution type, both of a nucleic acid probe complementary with a sequence in the case of A and a sequence in the case of T may be fixed to an array, or only a probe for one sequence may be fixed. Similarly, also in SNP of the 3-nucleotide substitution type and 4-nucleotide substitution type, probes corresponding to sequences in the case of respective nucleotides may be used, or only a probe corresponding to a sequence which is intended to be detected may be used. As a length of a nucleic acid probe, a length suitable for fixation to a substrate and hybridization may be appropriately selected, and the length may be shorter than that of a target nucleic acid. For example, the length may be about 3 to about 1000 bp, preferably about 10 to about 200 bp.

A target nucleic acid is a nucleic acid having a sequence consisting of sequences upstream and downstream of a site where SNP is present.

As a target nucleic acid which is subjected to hybridization with nucleic acid probes on an array, a test solution containing the nucleic acid may be used as it is, or a target sequence site may be amplified, for example, by PCR in advance, and excised, and used. Thereupon, a length of a target nucleic acid may be arbitrarily determined, but may be a length of, for example, around 30 to 500 bp by primer design. By adopting an appropriate length of a target nucleic acid, an efficacy of hybridization can be increased.

A substrate which can be used in the present invention may be a substrate to which a nucleic acid probe can be fixed, and may have a shape of a well, a plate having a groove or a planar surface, or a steric shape such as a sphere, made of a non-porous, hard or semi-hard material. The substrate can be manufactured, without limitation, of a silica-containing substrate such as silicon, glass, quartz glass and quartz, or a plastic or a polymer such as polyacrylamide, polystyrene, polycarbonate and the like.

In the array of the present invention, as a means for detecting the presence of a duplex generated as a result of hybridization between a nucleic acid probe fixed to a substrate and a target nucleic acid, an electrochemical method can be used, being not limited thereto.

Detection of a double-stranded nucleic acid by an electrochemical method may be performed using the known duplex recognizing substance. The duplex recognizing substance is, without limitation, Hoechster 33258, acridine orange, quinacrine, daunomycin, metallointercalater, bisintercalater such as bisacridine and the like, trisintercalater and polyintercalater can be used. Further, it is possible to modify these intercalaters with an electrochemically active metal complex, for example, ferrocene, biogen or the like. Alternatively, other known duplex recognizing substances may be used.

In a method of detecting a double-stranded nucleic acid by an electrochemical method, an electrode is provided on a substrate, and nucleic acid probes are fixed to this electrode. The electrode is not particularly limited, but can be formed of a carbon electrode such as graphite, glassy carbon, pyrolytic graphite, carbon paste, and carbon fiber, noble metal electrode such as platinum, platinum black, gold, palladium, and rhodium, an oxide electrode such as titanium oxide, tin oxide, manganese oxide, and lead oxide, a semiconductor electrode such as Si, Ge, ZnO, CdS, TiO₂ and GaAs, titanium or the like. These electrodes may be covered with an electrical conductive polymer, or may be covered with a single molecule membrane, or may be treated with other surface treating agent, if necessary.

Fixation of a nucleic acid probe may be performed by the known means. For example, a nucleic acid probe may be fixed to an electrode via a spacer by fixing a spacer to an electrode, and fixing a nucleic acid probe to the spacer. Alternatively, a spacer is bound to a nucleic acid probe in advance, and the probe may be fixed to an electrode via the spacer. Alternatively, a spacer and a nucleic acid probe may be synthesized on an electrode by the known means. In addition, for fixing a nucleic acid probe with a spacer, the spacer may be directly fixed to a treated or non-treated electrode surface with a covalent bond, an ionic bond or physical adsorption. Alternatively, a linker agent which assists fixing of a nucleic acid probe via a spacer may be used. In addition, an electrode may be treated with a blocking agent for preventing nonspecific binding of a test nucleic acid to an electrode together with a linker agent. In addition, a linker agent and a blocking agent used herein may be a substance for advantageously performing electrochemical detection.

Nucleic acid probes having different nucleotide sequences may be fixed to different electrodes via a spacer, respectively.

Further, like other general electrochemical detecting method, an array may be provided with counter electrodes and/or reference electrodes. When reference electrodes are arranged, for example, general reference electrodes such as silver/silver chloride electrode and mercury/mercury chloride can be employed.

Detection of a target nucleic acid by an array can be performed, for example, as follows. A nucleic acid component is extracted as a sample nucleic acid from a sample collected from a subject such as an individual such as an animal including a human, a tissue and a cell. The resulting sample nucleic acid may be subjected to treatment such as reverse transcription, elongation, amplification and/or enzyme treatment, if necessary. The sample nucleic acid which has been pre-treated as necessary is contacted with a nucleic acid probe immobilized on a nucleic acid probe immobilizing substrate, and a reaction is performed under the condition allowing for appropriate hybridization. A person skilled in the art can appropriately select such the appropriate condition depending on various conditions such as a kind of a nucleotide contained in a target sequence, a kind of a spacer and a nucleic acid probe set on a nucleic acid probe immobilizing substrate, a kind of a sample nucleic acid, and the state thereof.

A hybridization reaction may be performed, for example, under the following condition. As a hybridization reaction solution, a buffer having an ionic strength in a range of 0.01 to 5 and a pH in a range of 5 to 10 is used. To this solution, may be added dextran sulfate which is a hybridization promoter, as well as a salmon spermatozoon DNA, a bovine thymus DNA, and a surfactant such as EDTA. A sample nucleic acid is added thereto, and this is thermally denatured at 90° C. or higher. Immediately after denaturation of a nucleic acid, or after rapid cooling to 0° C., a nucleic acid probe immobilizing substrate is inserted into this solution. Alternatively, it is also possible to perform a hybridization reaction by adding dropwise the solution to a substrate.

During the reaction, a reaction rate may be enhanced by a procedure such as stirring and shaking. A reaction temperature is, for example, in a range of 10° C. to 90° C., and a reaction time is not shorter than 1 minute to around overnight. After the hybridization reaction, an electrode is washed. As a washing solution, for example, a buffer having an ionic strength in a range of 0.01 to 5 and a pH in a range of 5 to 10 is used. When target nucleic acids containing a target sequence is present in a sample nucleic acid, it is hybridized with nucleic acid probes to generate double-stranded nucleic acids on a substrate.

Subsequently, detection of a double-stranded nucleic acid is performed by electrochemical procedure. In a usual procedure, a substrate is washed after hybridization and then a duplex recognizing entity is acted on a double-stranded part formed on an electrode surface, and a signal generated therefrom is measured electrochemically.

A concentration of a duplex recognizing entity is different depending on a kind thereof, and generally, the entity is used in a range of 1 ng/mL to 1 mg/mL. Thereupon, a buffer having an ionic strength in a range of 0.001 to 5 and a pH in a range of 5 to 10 may be used.

Electrochemically measurement can be performed, for example, by applying a potential which is not lower than the potential at which a duplex recognizing entity reacts electrochemically, and measuring a reaction current value derived from the duplex recognizing entity. Thereupon, a potential may be scanned at a constant rate, or may be applied in a pulse manner, or a constant potential may be applied. Upon measurement, a current and a voltage may be controlled using a device such as a potentiostat, a digital multimeter and a function generator. Further, based on the resulting current value, a concentration of a target nucleic acid may be calculated from a calibration curve.

A sequence of a target nucleic acid detected by the hybridization reaction using the aforementioned array is a nucleotide sequence possessed by an individual to be tested, and a sample. Thereby, genotypes of selected SNPs in a nucleotide sequence contained in each sample can be determined.

After respective genotypes of an individual to be discriminated and a sample are determined, they are compared, and it is determined whether both are consistent or not. Further, based on a genotype frequency of used SNP, an existence ratio of genotype of the individual or the sample is calculated, and reliability of determination may be determined.

According to another aspect of the present invention, an individual discriminating test apparatus provided with the aforementioned array is provided. In the apparatus, a substrate-like array is suitably used, and is also referred to as chip herein. Further, according to another aspect of the present invention, a system for implementing an individual discriminating test by the individual discriminating test apparatus is provided.

The individual discriminating test apparatus of the present invention is provided with the array according to the present invention, a flow channel which is provided on a substrate of the array, and is provided along a flow direction of a drug solution or the air, a working electrode which is provided at a plurality of number on the substrate along the flow channel, and on which the probe is immobilized, counter electrodes for imparting an electric potential difference between the working electrodes, which are provided on an internal circumferential surface of the flow channel corresponding to the working electrodes, each being arranged so as to be situated on a first surface facing the substrate surface, reference electrodes for feed-backing a detected voltage to the working electrodes, which are provided on an internal circumferential surface of the flow channel corresponding to the working electrodes, each being arranged so as to be situated on a second surface facing the substrate surface, an inlet port which is opened in the flow channel, and flows a drug solution or the air in the flow channel from an upstream side of the flow channel, an outlet port which is opened in the flow channel, and flows out a drug solution or the air from the flow channel to a downstream side of the flow channel, and an injecting port for injecting test solution into the flow channel.

In addition, the individual discriminating test system according to the present invention is provided with the individual discriminating test apparatus, a first piping which is communicated with the inlet port, and supplying a drug solution or the air into the flow channel via the inlet port, a supply system provided with a first valve for controlling a flow rate of a drug solution or the air of the first piping, a second piping which is communicated with the outlet port, and discharges a drug solution or the air from the flow channel via the outlet port, a second valve for controlling a flow rate of a drug solution or the air of the second piping, a discharge system which is provided in the second piping and is provided with a pump for drawing up a drug solution or the air from the flow channel, a measuring system provided with a voltage applying unit for imparts an electric potential difference between the working electrode and the counter electrode, a temperature control system for controlling a temperature of the array, a control mechanism which controls the first valve of the supply system, the second valve and the pump of the discharge system, the voltage applying unit of the measurement system, and the temperature control system, detects an electrochemical reaction signal from the working electrode or the counter electrode, and stores this electrochemical reaction signal as measurement data, and a computer which imparts a control condition parameter to the control mechanism to control the control mechanism, and at the same time, executes nucleotide sequence analyzing treatment based on the measurement data, and determines consistency between a nucleotide sequence possessed by an individual and a nucleotide sequence possessed by a sample.

One embodiment of the individual discriminating test apparatus and system of the present invention will be explained below by referring to the drawings.

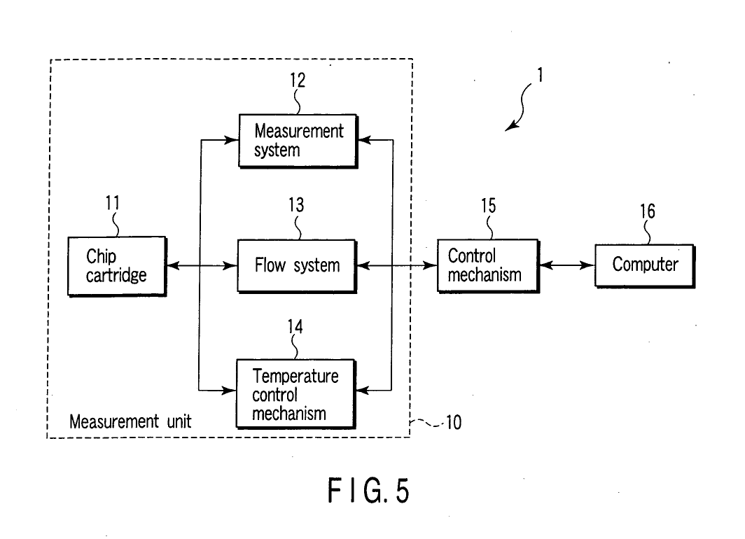

FIG. 5 is a conceptional view showing a whole construction of an individual discriminating test system in one embodiment of the present invention. As shown in FIG. 5, an individual discriminating test system 1 is constructed of a chip cartridge 11 (individual discriminating test apparatus), a measurement system 12 which is electrically connected to this chip cartridge 11, a flow system 13 which is physically connected to a flow channel provided in a chip cartridge 11, via an interface part, and a temperature control system 14 which controls a temperature of a chip cartridge 11.

These measurement system 12, flow system 13 and temperature control system 14 are controlled by a control mechanism 15. A control mechanism 15 is electrically connected to a computer 16, and a control mechanism 15 is controlled by a program provided in this computer 16. In the present embodiment, the chip cartridge 11, the measurement system 12, the flow system 13 and the temperature control system 14 are referred to as a measurement unit 10.

A printed board 22 packaged with a chip 21 on which a nucleic acid probe is immobilized is attached to a chip cartridge 11, which is used. A nucleic acid probe is immobilized on a working electrode of a chip 21. A sample (test solution) which is introduced into a cell of a chip 21 contains a nucleic acid to be tested. The individually discriminating test apparatus of this embodiment determines whether a target nucleic acid is contained in a sample (test solution) or not by hybridizing a target nucleic acid with a nucleic acid probe, and monitoring the presence or the absence of the reaction after introduction of a buffer and an intercalator.

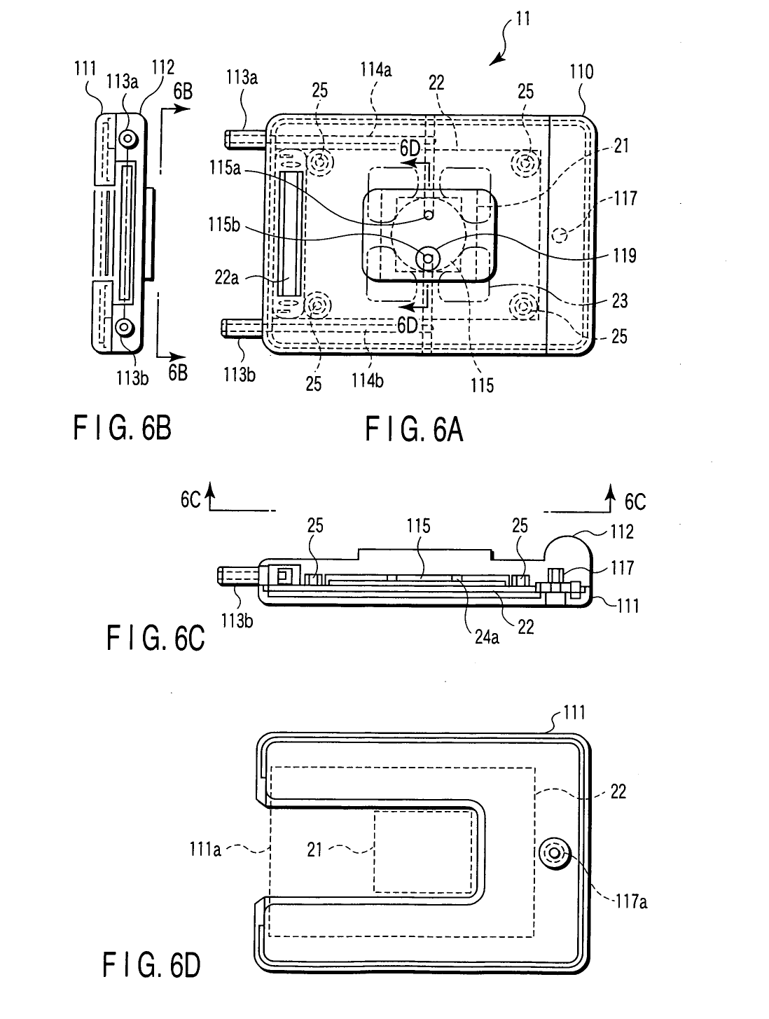

FIGS. 6A to 6D are views showing a detailed construction of a chip cartridge 11. FIG. 6A shows a view seen from an upper surface, FIG. 6B shows a view seen in an B-B direction, FIG. 6C shows a partial perspective cross-sectional view seen in a C-C direction, and FIG. 6D shows a view of a support 111 which is one constitutional element of a chip cartridge 11 seen from a back in an D-D direction. A chip cartridge body 110 consists of a support 111 which supports a printed board 22 from a lower side, and a chip cartridge upper lid 112 for holding, fixing and supporting a printed board 22 from an upper side, in conjunction with this support 111.

Two openings are provided on a side part of a chip cartridge upper lid 112, and an interface part 113a is connected to one of openings, and an interface part 113b is connected to the other opening. These interface parts 113a and 113b function as an interface for a flow system 13 and a chip cartridge 11. In an interior of these interface parts 113a and 113b, flow channels 114a and 114b are provided, respectively. A drug solution or the air from an upper stream side of a flow system 13 is flown into an interior of a chip cartridge 11 via a flow channel 114a. A sample, a drug solution and the air in a hip cartridge 11 are flown out to a downstream side of a flow system 13 via a flow channel 114b.

In FIGS. 6A to 6C, flow channels 114a and 114b are indicated with a broken line. These flow channels 114a and 114b are communicated with an interior of a chip cartridge upper lid 112 from interface parts 113a and 113b, and are further communicated with a cell 115. A cell 115 is a region provided for generating an electrochemical reaction between a chip 21 and various solutions which are introduced into this chip 21. This 115 is defined by a closed space region surrounded by a chip 21, a sealing material 24a, and a chip cartridge upper lid 112 when four corners of a printed board 22 packaged with a chip 21 are fixed to a chip cartridge upper lid 112 of this chip cartridge 11 with a substrate fixing screw 25. In the state where a printed board 22 packaged with a chip 21 is fixed to a chip cartridge upper lid 112, a printed board 22 is retained with a support 111 and a chip cartridge upper lid 112, holding a sealing material 24a. Further, a chip cartridge upper lid 112 is fixed with an upper lid fixing screw 117. Thereby, injection and discharge pathways for various drug solutions and the air which are communicated with a flow channel 114b from a flow channel 114a via a cell 115 are defined. A chip 21 is sealed to a printed board 22 with a sealing resin 23.

A chip cartridge upper lid 112 is situated on an upper side of a cell 115 provided with an inlet port 116a and an outlet port 116b. A flowing port in 116a penetrates from a side surface to a bottom surface of a chip cartridge upper lid 112, and is opened on a bottom surface of a chip cartridge upper lid 112 at a cell pore part 115a. A outlet port 116b penetrates from another side surface to a bottom surface of a chip cartridge upper lid 112, and is opened on a bottom surface of a chip cartridge upper lid 112 at a cell pore part 115b. By connection of an inlet port 116a to a flow channel 114a, and of an outlet port 116b to a flow channel 114b, a flow channel 114a and a cell 115, and a flow channel 114b and a cell 115 are communicated.

An electric connecter 22a is provided at a position which is a printed board 22 surface and is spaced from a cell 115. An electric connector 22a is electrically connected to a lead frame of a substrate body of a printed board 22. In addition, this lead frame of a substrate body is electrically connected to various electrodes of a chip 21 with a lead. By connecting a terminal of a measurement system 12 to this electric connector 22a, an electric signal obtained in a chip 21 can be outputted to a measurement system 12 via a predetermined terminal provided at a predetermined position of a printed board 22, and further, via an electric connector 22a.

As shown in FIG. 6D, a support 111 has a U-shape, and a notch part 111a is provided at a center thereof. This notch part 111a has a shape smaller than a printed board 22 and larger than a chip 21. Thereby, a temperature control system 14 is contact-disposed on a chip 21 without via a support 111 while the function of supporting a printed board 22 with a support 111 is retained. 117a is a screw pore, and an upper lid fixing screw 117 is fixed therein.

As a temperature control system 14 for regulating a temperature of a chip 21, for example, a Peltier element is used. Thereby, temperature control of ±0.5° C. is possible. A reaction of a nucleic acid is generally performed at a temperature range relatively near room temperature. Therefore, temperature control only with a heater is poor in stability. In addition, since it is necessary to control a reaction of a nucleic acid by a temperature profile, another cooling mechanism becomes to be necessary. In this respect, in a Peltier element, since any of heating and cooling is possible by changing a direction of a current, the element is optimal.

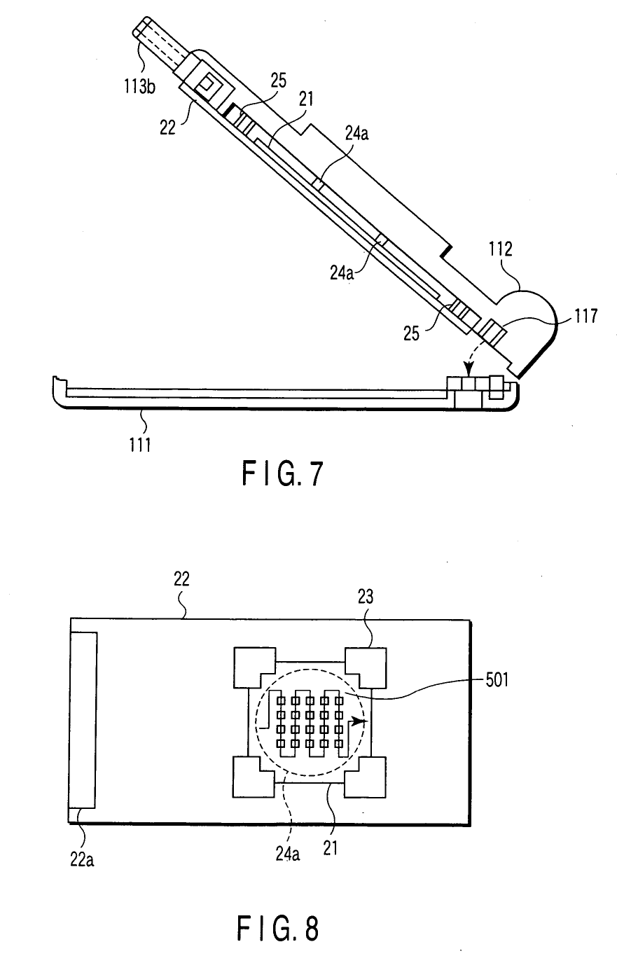

FIG. 7 is a view showing a support 111 and a chip cartridge upper lid 112 before fixation with an upper lid fixing screw 117. As shown in FIG. 7, four corners of a printed board 22 packaged with a chip 21 are fixed on a chip cartridge upper lid 112 with a substrate fixing screw 25. A sealing material 24 a is integrated into a chip cartridge upper lid 112. Therefore, a cell 115 surrounded by a sealing material 24a and a chip cartridge upper lid 112 is defined on a chip 21. Further, a chip cartridge upper lid 112 is fixed on a support 117 with an upper lid fixing screw 117, which is used. In addition, a substrate fixing screw 25 may fix a subject from a back side or from a surface side of a printed board 22. Like this, by fixing a printed board 22 on a chip cartridge upper lid 112, adherability between the chip 21, the sealing material 24a and the chip cartridge upper lid 112 can be assuredly retained.

FIG. 8 is a view showing a detailed construction of a printed board 22 packaged with a chip 21. As shown in FIG. 8, a chip 21 is sealed on a printed board 22 with a sealing resin 23. On a chip 21, a working electrode 501 is provided. This working electrode 501 is provided one by one along a direction of flow of a drug solution and the air indicated by an arrow of FIG. 8. A direction of flow of a drug solution and the air is defined by closure with a chip cartridge upper lid 112 and the sealing material 24a, leaving a space along a direction indicated by an arrow around a working electrode 501 on a chip 21. A region shown by a broken line is a region on which a sealing material 24a is disposed. A plurality of working electrodes 501 are arranged so as to be accommodated in a region indicated by this broken line.

An electric connected 22a is disposed at an end of a printed board 22. A working electrode 501 of a chip 21 and an electric connector 22a are electrically connected with a lead frame provided on a surface of a printed board 22. Various electrodes of a chip 21 and a measurement system 12 can be electrically connected to an electrical connector 22a by connecting a signal interface of a measurement system 12.

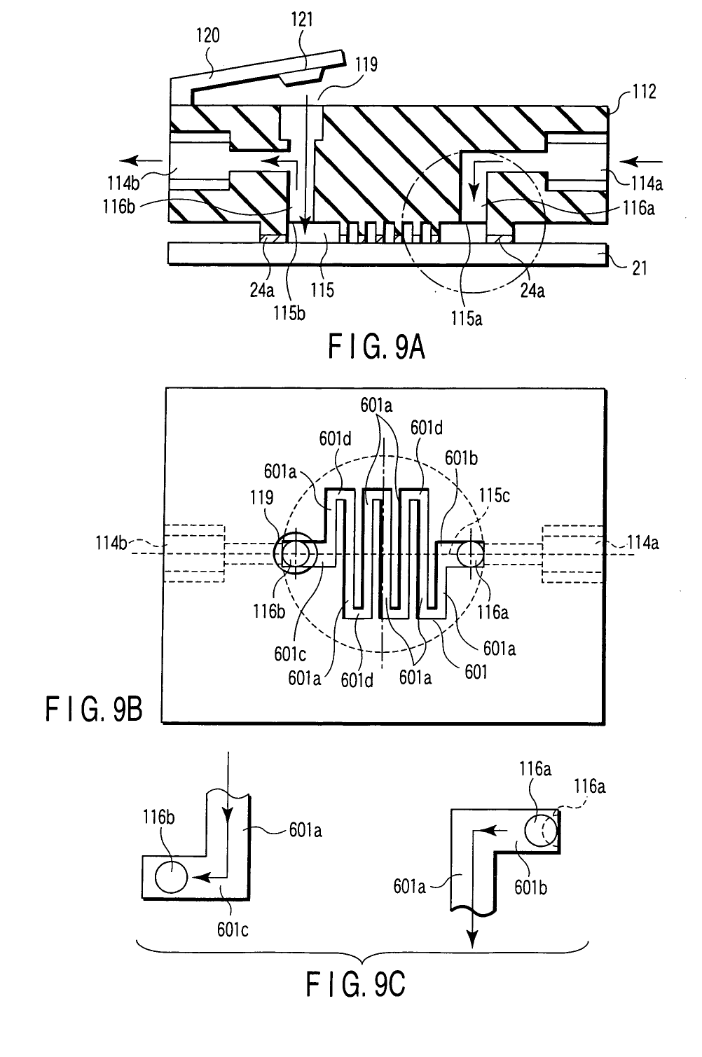

FIG. 9A is a cross-sectional view of a cell 115 and a drug solution supply system communicating with a cell 115 shown in FIG. 6A seen in a D-D direction, and FIG. 9B is a top view of a vicinity of a cell 115. As shown in FIG. 9A, a flow channel-like convex part 112a having a height of d42 is provided on a bottom of a chip cartridge upper lid 112. A sealing material 24a is printed on this flow channel-like convex part 112a in advance, for example, by screen printing, and the part is integrally formed with a sealing material 24a. Thereby, a cell 115 can be defined without positioning a sealing material 24a and a chip cartridge upper lid 112, and a step of assembling a cell 115 becomes simple. A sealing material 24a is fixed between a flow channel-like convex part 112a and a chip 21. Thereby, a closed space is defined between a chip cartridge upper lid 112 and a chip 21. This closed space is a cell 115 as a reaction chamber for generating an electrochemical reaction between a sample or a drug solution and a probe. A bottom of a cell 115 is defined by a chip 21. A side surface of a cell 115 is defined by a flow channel-like convex part 112 provided on a chip cartridge 112, and a side part of a sealing material 24a. An upper surface of a cell 115 is defined by a site of a chip cartridge 112, in which a flow channel-like convex part 112a is not provided. Thereby, a closed space in which entities other than cell pore parts 115a and 115b are closed is defined, and liquid tight between a chip 21 and a lid 120 is retained. A height of this cell 115 is set at about 0.5 mm. Herein, the height is set at around 0.5 mm, being not limited thereto, and it is desirable to set in a range of 0.1 to 3 mm.

A cell 115, when seen from an upper side, has a shape in which an elongate flow channel 601 is arranged as shown in FIG. 9B. In FIG. 9B, one flow channel 601 having the same channel width is provided from a cell pore part 115a on an inlet port 116a side toward a cell pore part 115b. This one flow channel 601 consists of a detection flow channel 601a, port collecting flow channels 601b and 601c, and a flow channel connecting flow channel 601d. A detection flow channel 601a is a plurality of flow channels in which a working electrode 501 is arranged. A port connecting flow channel 601b connects a detection flow channel 601a nearest a cell pore part 115 a to a cell pore part 115a. A pore connecting flow channel 601c connects a detection flow channel 601a nearest a cell pore part 115b to a cell pore part 15b. A flow channel connecting flow channel 601d connects ends of detection flow channels 601a which are adjacent to each other, to define a direction of flowing of a drug solution or the air into a plurality of detection flow channels 601a in one direction. Thereby, a drug solution or the air which has flown in a certain detection flow channel 601a is flown into a flow channel connecting flow channel 601d, and further, is flown into another detection flow channel 601a adjacent in the same direction. In addition, all of flow channels 601a to 601d have the same channel width and cross-section, and the channel width is desirably 0.5 to 10 mm.

In FIG. 9B, a region which is surrounded by a broken line and in which a flow channel 601 is not formed is a region in which a flow channel-like convex part 112a and a sealing material 24a are provided, and a chip 21 and a sealing material 24a are contacted. A region in which a flow channel 601 is formed is a region in which a flow channel-like convex part 112a and a sealing material 24a are not provided. An inlet port 116a and an outlet port 116a extend upwardly from an upper side of a cell 115 to a predetermined height, respectively, in a direction approximately vertical to a cell bottom surface. An inlet port 116a and an outlet port 116b are further bent in their flow channel from a center of a cell 115 toward a direction far away from each other, and are connected to flow channels 114a and 114b, respectively.

An outlet port 116b extends to a predetermined height in a direction approximately vertical to a cell bottom surface, and further, is bent approximately orthogonally in a direction far away from a center of a cell 115, and is branched into two pathways at the bending position. One pathway penetrates to a surface of a chip cartridge upper lid 112, and is communicated with an injecting port 119. Thereby, a sample injected through an injecting port 119 is introduced into a cell 115 through an outlet port 116b. A central axis of an injection port 119 and that of an outlet port 116b are approximately consistent, and an aperture diameter of an injection port 119 is set to be greater than an aperture diameter of a flowing port 116b. In addition, it is provided in vicinity of an injection port 119, and an injection port 119 can be covered with a lid 120. Thereby, without utilizing an injection port 119, when a drug solution is circulated in a flow channel 114b from a flow channel 114a to a flow channel 114b via a cell 115, a drug solution can be prevented from flowing out through an injection port 119, and a pathway of a drug solution can be maintained. In addition, a sealing material 121 is provided on a lid 120, and by sealing an injection port 119, slight leakage of a drug solution can be prevented. In an example of FIG. 9A, although not particularly shown, when a sealing material 121 having such a depth that a pathway to an injection port 119 is completely clogged, leaving only a pathway connected to a flow channel 114b from an outlet port 116b is used, retention of a drug solution or the air on an injection port 119 can be reduced.

By the above construction, a drug solution can be flown in an order of a flow channel 114a, an inlet port 116a, a cell 115 (flow channel 601), an outlet port 116b and a flow channel 114b in a direction shown by an arrow in FIG. 9A. In addition, a sample is injected through an injection port 119, and is introduced into a cell 115 through an outlet port 116b in an arrow direction. Therefore, a sample is injected from a flowing out side, and an injection pathway is conversely set relative to a flow of a supply of a drug solution. Thereby, in a washing step, a washing efficacy of a sample can be enhanced.

FIG. 9C is a view showing an optimal positional relationship between an inlet port 116a, an outlet port 116b and a flow channel 601. An external circumference of an inlet port 116a is contacted with an external circumference of a port connecting flow channel 601b. In addition, an external circumference of an outlet port 116b is apart from an external circumference of a port connecting flow channel 601c. Thereby, upon drug solution or air flowing in, remaining of a drug solution or remaining of the air which is easily generated in a vicinity of a port corner of an inlet port 116a can be reduced, and at the same time, a scatter in a flowing rate generated at a port corner of an outlet port 116b upon drug solution or air flowing out can be reduced, and air remaining can be reduced. In addition, as shown by a broken line in the same figure, when an inlet port 116a is configured to be protruded from a port connecting flow channel 601b by overlapping between an external circumference of a port connecting flow channel 601b and an external circumference of inlet port 116a, the similar effect can be obtained. Of course, a positional relationship between an inlet port 116a and a flow channel 601 of an outlet port 116b is not limited to the relationship shown in FIG. 9C. In an inlet port 116a side, three cases of the case where circumferences of both are contacted, the case where they are overlapped, and the case where they are separated are considered in connection to a port connecting flow channel 601b, and also in an outlet port 116b side, three cases of the case where circumferences of both are contacted, the case where they are overlapped, and the case where they are separated are considered in connection to a port connecting flow channel 601c.

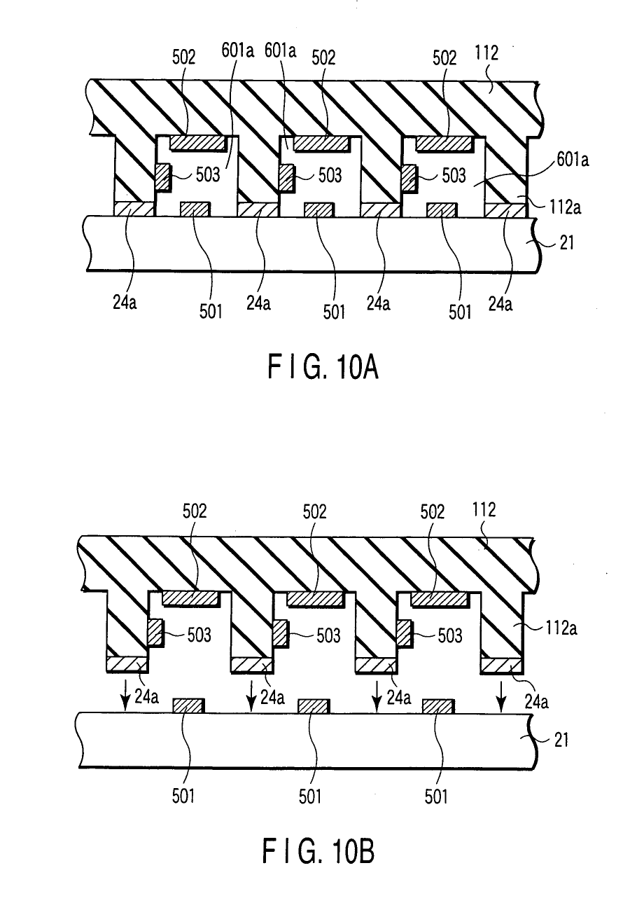

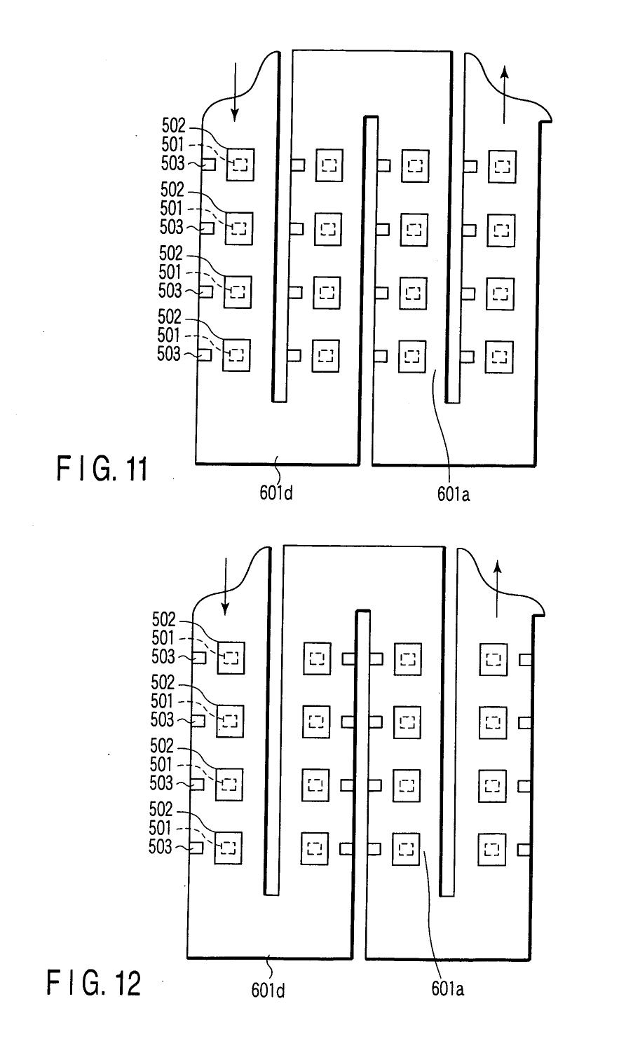

FIGS. 10 and 11 are a view showing a detailed construction of a cell 115. FIG. 10A is a cross-section in which a cell is cut with a straight line connecting cell pore parts 115a and 115b, FIG. 10B is a view showing appearance in which a chip cartridge upper lid 112 is fixed to a chip 21, and FIG. 11 is a top view of a cell 115. As shown in FIG. 10A, a plurality of detection flow channels 601a are formed at approximately the same interval. When a drug solution or the air is flown in a cross-section of a detection flow channel 601a shown on a left side of FIG. 10A from a rear side to a front side, it is flown in a central detection flow channel 601a in a reverse direction, that is, is flown from a front side to a rear side, and it is flown in a detection flow channel 601a shown on a left side in a further reverse direction, that is, is flown in a direction from a rear side to a front side. Like this, directions of flow of a drug solution or the air of adjacent detection flow channels 601a are reverse. When these detection flow channels 601a are cut with a cross-section vertical to a direction of flow of a drug solution or the air, all form the same oblong cross-sectional shape, and electrode arrangement is the same.

A bottom surface of a detection flow channel 601a is defined by a chip 21. Each one of a working electrode 501 is formed on each bottom surface of a detection flow channel 601a. A side surface of a detection flow channel 601a is defined by a flow channel-like convex part 112a which is provided in a convex manner from a chip cartridge upper lid 112, and a sealing material 24a. A reference electrode 503 is fixed on a side surface of this flow channel, that is, a side surface of a flow channel-like convex part 112a, respectively, to a predetermined height from a flow channel bottom. Like this, a plurality of reference electrodes 503 are situated on a plane which is parallel to a chip surface and faces with a chip surface and the plane is situated on a plane higher than a plane on which a working electrode 501 is provided. An upper side of a detection flow channel 601a is defined by a bottom surface of a chip cartridge upper lid 112 on which a flow channel-like convex part 112a is not provided. Each counter electrode 502 is fixed on an upper side of this flow channel. Like this, a plurality of counter electrodes 502 are situated on a plane which is parallel to a chip bottom and faces with a chip surface, and the plane is situated on a plane higher than a plane on which a working electrode 502 or a reference electrode 503 is provided. Like this, a working electrode 501, a counter electrode 502 and a reference electrode 503 are three-dimensionally arranged on different planes, respectively.

A sealing material 24a is immobilized on a flow channel-like convex part 112a of a chip cartridge upper lid 112 with printing in advance. Therefore, when a cell 115 is assembled, a chip cartridge upper lid 112 integrated with a sealing material 24a is pushed against a chip 21 in a direction shown by an arrow of FIG. 10B. Thereby, a flow channel 601 having a closed periphery as shown in FIG. 10A is defined between a chip cartridge upper lid 112 and a chip 21 via a sealing material 24a.

As shown in FIG. 11, three electrodes consisting of a working electrode 501, a counter electrode 502 and a reference electrode 503 are arranged in each detection flow channel 601a at an equal interval in a direction of flow of a drug solution or the air which is indicated by arrows. The three electrodes are arranged on planes vertical to a direction of flow of a drug solution or the air, respectively.

In an example of FIG. 11, arrangement in which a positional relationship between a working electrode 501, a counter electrode 502 and a reference electrode 503 is the same matrix state when seen from an upper side irrespective of a direction of a flow channel, being not limited thereto. As shown in FIG. 12, structures of a flow channel cross-section in adjacent detection flow channels 601a may be reversed left and right along a direction of flow of a drug solution or the air. In this case, a counter electrode 502 is arranged on a side surface on a right side of a flow channel toward a flow direction, in any detection flow channel 601a. Thereby, three electrode arrangement all having the same shape in a flow direction of a drug or the air can be realized. Also regarding a working electrode 501 and a counter electrode 502, when it is not arranged in a left and right symmetric position in a cross-section, it can be arranged at a left and right reversed position in adjacent detection flow channels 601a as in this reference electrode 503.

Like this, each one of a working electrode 501, a counter electrode 502 and reference electrode 503 are provided as one set of three electrodes in the same cross-sectional shape flow channel along a direction of flow of a drug solution or the air, and a construction is such that a positional relationship of these three electrodes is the same, and a flow channel shape is the same. When seen from a working electrode 501, directions to a flow channel bottom surface, a side surface and an upper surface relative to a working electrode 501, and positional relationships from a working electrode 501 to corresponding counter electrode 502 and reference electrode 503 are the same. Thereby, uniformity of property of an electrochemical signal detected by each of three electrodes is improved. As a result, detection reliability is improved.

Herein, a counter electrode 502 and a reference electrode 503 are arranged so as to be separated relative to a corresponding working electrode 501, respectively, being not limited thereto. A counter electrode 502 or a reference electrode 503 may have a construction that pluralities of electrodes are connected in any case. In that case, a region nearest each working electrode in each electrode functions as a counter electrode or a reference electrode. In addition, a cross-sectional shape of a flow channel is not limited to the aforementioned construction of FIG. 10A.

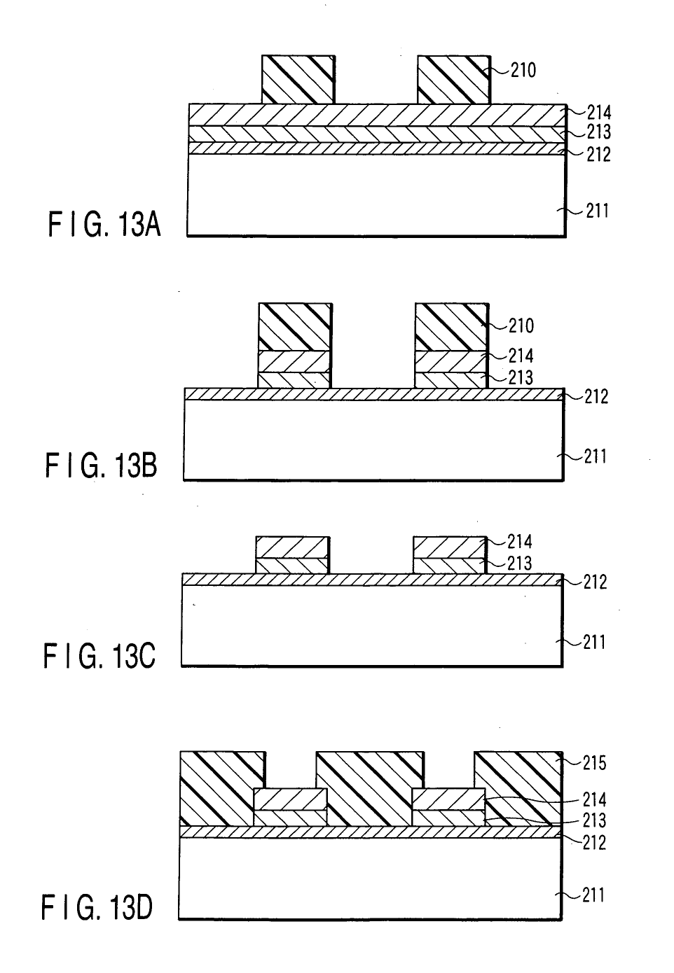

Then, a process for manufacturing the aforementioned chip 21 and printed board 22 will be explained in line with a step cross-sectional view of FIG. 13. A silicon substrate 211 is washed, and a silicon substrate 211 is heated to form a thermally oxidized film 212 on a surface of a silicon substrate 211. In place of a silicon substrate 211, a glass substrate may be used. Then, a Ti film 213 is formed on a whole substrate, for example, at a film thickness of 50 nm, and then, an Au film 214 is formed on a whole substrate, for example, at a film thickness of 200 nm by sputtering. Herein, it is preferable that an Au film 214 has its crystalline plane direction of <111>orientation. Then, a photoresist film 210 is patterned (FIG. 13A) so as to protect a region which becomes an electrode or a wiring later, and an Au film 214 and Ti film 213 are etched (FIG. 13B). In the present embodiment, a KI/I2 mixed solution was used for etching an Au film 214, and a NH₄OH/H₂O₂ mixed solution was used for etching Ti. For etching an AU film 214, there are a method using diluted aqua regia, and a method of removal with ion milling. For etching a Ti film 213, similarly, a method of wet etching treatment using hydrofluoric acid or buffered hydrofluoric acid, and a method of dry etching using plasma derived from a CF₄/O₂ mixed gas can be applied.

Then, a photoresist film 210 is removed by oxygen ashing (FIG. 13C). A step of removing a photoresist film 210 can be performed by using a solvent, using a resist stripper, or using an oxygen ashing step jointly.

Then, a photoresist 215 is coated on a whole surface, and this is patterned so as to open an electrode part and a bonding pad (FIG. 13D). Thereafter, hard baking is performed, for example, at 200° C. for 30 minutes in a clean oven. In a method of hard baking, a hot plate can be used or treating condition can be appropriately used. Herein, a photoresist film 215 was selected as a protective film, but in addition to a photoresist, an organic film such as polyimide BCB (benzocyclobutene) and the like can be also used. Alternatively, an inorganic film such as SiO, SiO₂ and SiN may be used. In that case, a film may be formed by opening a photoresist so as to protect an electrode part, depositing SiO or the like, and protecting a region other than an electrode part by a lift off method, or forming SiN or the like on a whole surface, forming a pattern of a photoresist film 215 so as to open only an electrode part, removing a SiN film or the like on an electrode by etching, and finally, peeling a photo resist film 215.

Then, chipping is performed by dicing finally in order to clean an electrode surface, treatment with a CF₄/O₂ mixed plasma is performed. Thereby, a chip 21 is obtained. This chip 21 is mounted on a printed board 22 packaged with an electric connecter 22. A bonding pad of a chip 21 and a lead wiring on a printed board 22 are connected by wire bonding. Thereafter, a wire bonding part is protected using a sealing resin 23. By the above step, a printed board 22 packaged with a chip 21 can be manufactured.

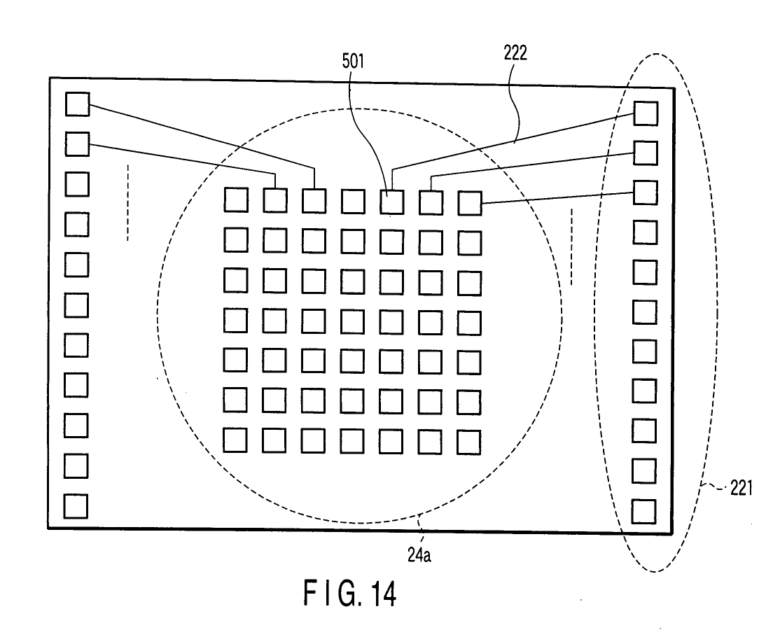

A top view of a manufactured chip 21 is shown in FIG. 14. As shown in FIG. 14, more than one working electrodes 501 are provided in a vicinity of a center of a chip surface. In addition, a region in which a working electrode 501 is formed is used so as to be accommodated in a region in which a sealing material 24a indicated by a broken line is formed. In addition, a bonding pad 221 is arranged at a periphery part of a chip. Each working electrode 501 is connected to a bonding pad 221 with a wiring 222. Although not shown in this FIG. 14, a periphery part in which a bonding pad 221 is formed is sealed with the aforementioned sealing resin 23.

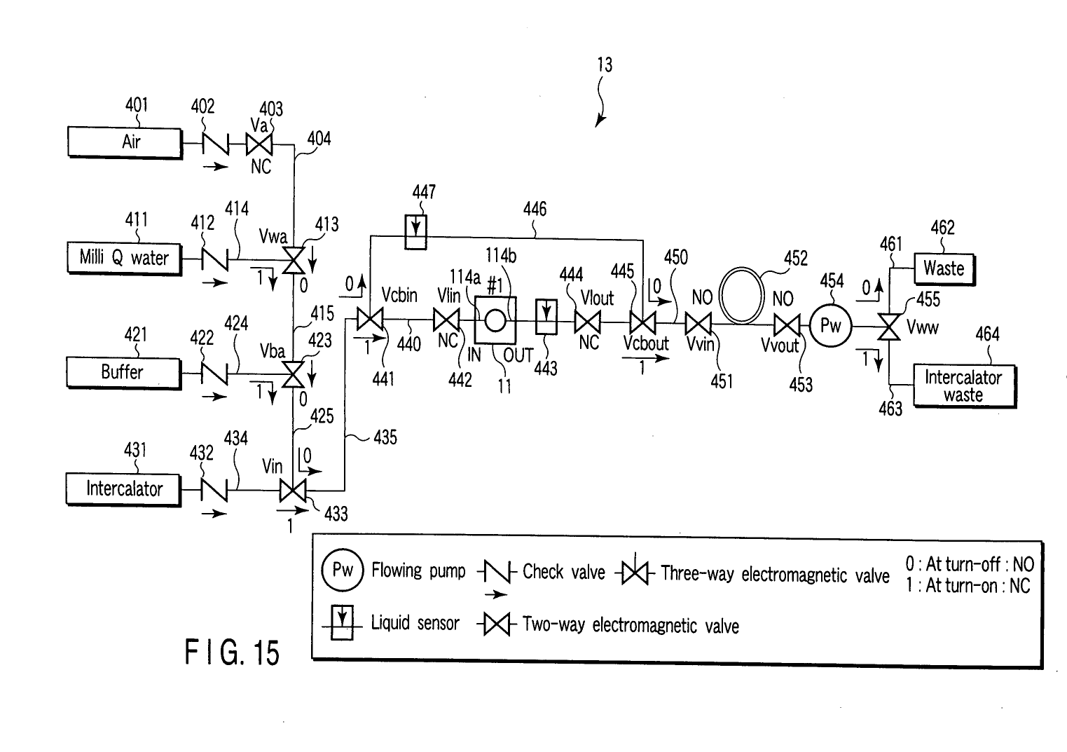

Then, one example of a specific construction of a flow system 13 is explained using FIG. 15. This flow system 13 is roughly classified into a supply system provided on a flow channel 114a side of a chip cartridge 11, and a discharge system provided on a flow channel 114b side. An air supply source 401 is connected to a most upstream of a piping 404. On a downstream side of this air supply source 401, a check valve 402 for preventing reverse flow of a drug solution other than the air to an air supply source 401 via a piping 404 is provided, and on a further downstream side, a two-way electromagnetic valve 403 (Va) is provided. Thereby, a flow rate of the air flowing into a chip cartridge 11 from a piping 404 is controlled.

A Milli Q water supply source 411 accommodating Milli Q water as one of drug solutions is connected to a piping 414. On a downstream side of this Milli Q water supply source 411, a check valve 412 for preventing reverse flow of a drug solution or the air other than Milli Q water to a Milli Q water supply source 411 is provided, and on a further downstream side, a three-way electromagnetic valve 413 (Vwa) is provided. This three-way electromagnetic valve 413 switches between communication of a piping 404 with a piping 415, and communication of a piping 414 and a piping 415. That is, at turn-off of a three-way electromagnetic valve 413, a piping 404 is communicated with a piping 415, and at turn-on, a piping 414 is communicated with a piping 415. Thereby, supply of the air and Milli Q water to a piping 415 can be switched.

A buffer supply source 421 accommodating a buffer as one of drug solutions is connected to a piping 424. On a downstream side of this buffer supply source 421, a check valve 422 for preventing reverse flow of a drug solution other thane a buffer or the air to a buffer supply source 421 is provided, and on a further downstream side, a three-way electromagnetic valve 423 (Vba) is provided. This three-way electromagnetic valve 423 switches between communication of a piping 424 with a piping 425, and communication of a piping 415 with a piping 425. That is, at turn-off of a three-way electromagnetic valve 423, a piping 415 is communicated with a piping 425, and at turn-on, a piping 424 is communicated with a piping 425. Thereby, supply of a buffer to piping 425, and supply of the air or Milli Q water can be switched.

An intercalator supply source 431 accommodating an intercalator as one of drug solutions is connected to a piping 434. On a downstream side of this intercalator supply source 431, a check valve 432 for preventing reverse flow of a drug solution other than a intercalator or the air to an intercalator supply source 431 is provided, and on a further downstream side, a three-way electromagnetic valve 433 (Vin) is provided. This three-way electromagnetic vale switches between communication of a piping 434 with a piping 435, and communication of a piping 425 with a piping 435. That is, at turn-off of a three-way electromagnetic valve 433, a piping 425 is communicated with a piping 435, and at turn-on, a piping 434 is communicated with a piping 435. Thereby, supply of an intercalator to a piping 435, and supply of the air, Milli Q water or a buffer can be switched.

As described above, in an air or drug supply system, by controlling a two-way electromagnetic valve 403 and three-way electromagnetic valves 413, 423 and 433, supply of the air, Milli Q water, and a drug solution such as a buffer and an intercalator to a chip cartridge 11 via a piping 435 can be switched, and a flow rate of the air or these drug solutions to be supplied can be controlled.

With an upstream side of a piping 435, the aforementioned three-way valve 433 is communicated, and with its downstream side, a three-way electromagnetic 441 (Vcbin) is communicated. By a three-way electromagnetic valve 441, a piping 435 can be branched into a piping 440 and a bypass piping 446. A three-way electromagnetic valve 441 switches as follows: at turn-off, a piping 435 is communicated with a bypass piping 466, and at turn-on, a piping 435 is communicated with a piping 440. In addition, a three-way electromagnetic valve 445 switches as follows: at turn-off, a bypass piping 446 is communicated with a piping 450, and at turn-on, a piping 440 is communicated with a piping 450. By these three-way electromagnetic valves 441 and 445, supply of various drug solutions and the air can be switched between a bypass piping 446 and a piping 440.

In a piping 440, in an order towards a downstream side which is seen from a three-way electromagnetic valve 441, a two-way electromagnetic valve 442 (Vlin), a chip cartridge 11, a liquid sensor 443, a two-way electromagnetic valve 444 (Vlout), and a three-way electromagnetic valve 445 (Vcbout) are provided. With a two-way electromagnetic valve 442, a flow channel 114a corresponding to a flowing in system of a chip cartridge 11 is communicated, and with a two-way electromagnetic valve 444 side, a flow channel 114b corresponding to a flowing out system of a chip cartridge 11 is communicated. Thereby, a drug solution or the air is supplied to a flowing in system of a chip cartridge 11 via a piping 440, and these drug solution and air can be discharged from a flowing out system of a chip cartridge 11. In addition, by two-way electromagnetic valves 442 and 444, a flow rate of a drug solution or the air in this passway for a flowing solution and a discharging solution can be controlled. In addition, by a liquid sensor 443, a flow rate of a drug solution which is flown into a chip cartridge 11, or a flow rate of a drug solution discharged from a chip cartridge 11 can be monitored.

In a piping 450, in an order toward a downstream side which is seen from a three-way electromagnetic valve 445, a two-way electromagnetic valve 451 (Vvin), a reduced pressure region 452, a two-way electromagnetic valve 453 (Vout), a flowing pomp 454, and a three-way electromagnetic valve 455 (Vww) are provided. Two-way electromagnetic valves 451 and 453 prevent reverse flow of a drug solution or the air in a passway around a reduced pressure region 452. A flowing pomp 454 consists of a tube pump, and is characterized in that it is provided in a discharge system on a flowing out side (downstream side) when seen from a chip cartridge 11. That is, since a drug solution is not contacted with a mechanism other than a tube wall by using a tube pump, this is preferable from a viewpoint of pollution prevention. In addition, by performing supply to a drug solution or the air to a chip cartridge 11 and discharge from a drug solution or the air from a chip cartridge 11 by suction action, not only replacement between a drug solution and the air in an interior of a chip cartridge 11 can be performed smoothly, but also, even when a piping is loosened accidentally, or when a chip cartridge 11 is slipped from a piping 440, liquid leakage does not occur. Thereby, stability of apparatus mounting is improved.

Of course, a pump is provided in a piping on an upstream side of a chip cartridge 11, and this pump can be configured to pump the air or a drug solution to a chip cartridge 11. A pump is not limited to a tube pump, but a syringe pump, plunger pump, a diaphragm pump, and a magnetic pump may be used.

A three-way electromagnetic valve 455 switches so as to communicate a piping 450 with a piping 461 at turn-off, and communicate a piping 450 with a piping 463 at turn-on. A waste tank 462 is provided in a piping 461, and an intercalator waste tank 464 is provided in a piping 463. Thereby, a drug solution such as Milli Q water and a buffer other than an intercalator can be guided to a waste tank 462 by switching with a three-way electromagnetic valve 455, and an intercalator can be guided to an intercalator waste tank 464. Thereby, it becomes possible to fractionation-recover an intercalator.

In addition, electromagnetic valves may be connected with a piping such as a teflon tube, and the present embodiment can be configured of a manifold structure in which an electromagnetic valve and a flow channel are integrated on an upper stream side and a downstream side relative to a chip cartridge 11, respectively. Thereby, since a volume in a piping is reduced, an amount of a necessary drug solution can be considerably decreased. In addition, since drug solution flow is stabilized in a piping, reproductivity and stability of detection result are improved.

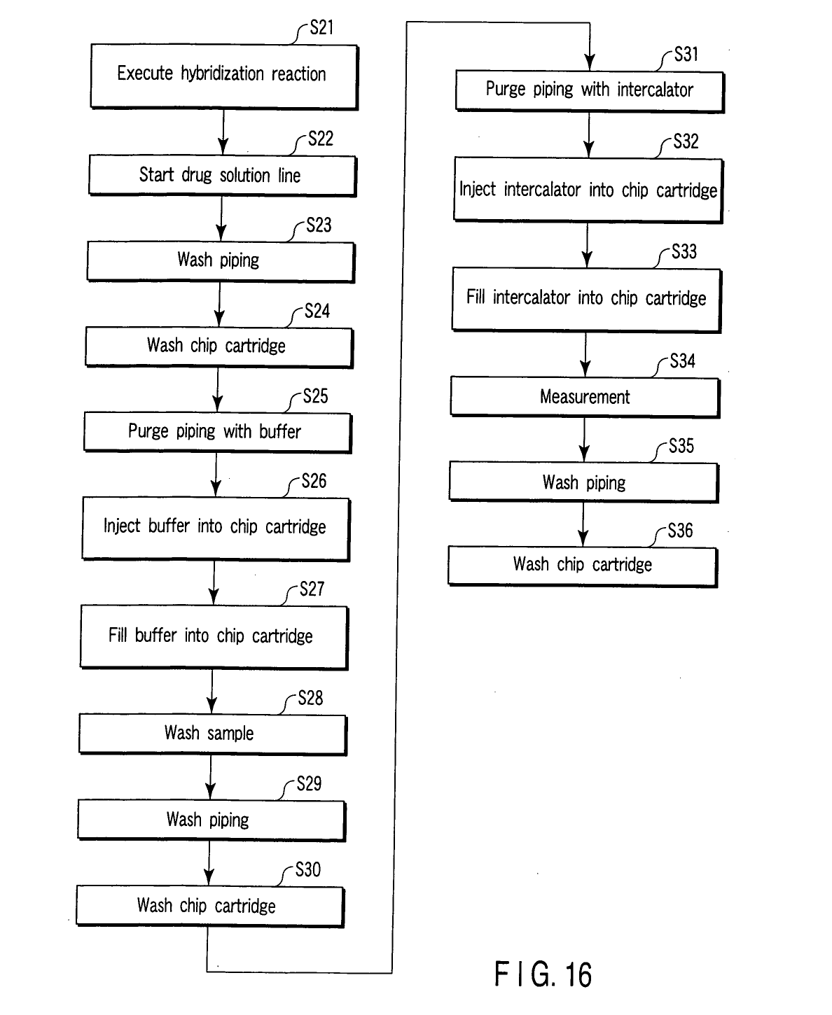

A flowing step using a flow system 13 shown in FIG. 15 will be explained using a flowchart of FIG. 16. First, a hybridization reaction between a nucleic acid probe immobilized on a working electrode 501 and a sample is performed in a cell 115 (s21). For this hybridization reaction, for example, a temperature control system 14 is controlled so that a bottom surface of a chip cartridge 11, that is, a bottom surface of a printed board 22 becomes around 45° C., and a temperature is retained, for example, for 60 minutes.

Parallel with this hybridization reaction, a drug solution line is started (s22). Specifically, by controlling three-way electromagnetic valves 441 and 445, a bypass piping 446 side is utilized, and by turning on a three-way electromagnetic valve 433, an intercalator is supplied from an intercalator supply source 431, for example, for around 10 seconds. A three-way electromagnetic valve 455 is turned on, and an intercalator from a piping 450 is accommodated in an intercalator waste tank 464. Then, an intercalator and the air are alternately introduced into a bypass piping 446 from a piping 435, repeatedly, for example, for 5 seconds. Then, only the air is introduced into a bypass piping 446 from a piping 435. At this stage, a waste is switched to a waste tank 462. A buffer is introduced into a bypass piping 446 from a buffer supply source 421. Thereafter, Milli Q water and the air are alternately introduced into a bypass piping 446 from a piping 435, repeatedly, for example, for each 5 seconds.

When starting of this drug solution line is completed, and a hybridization reaction is completed, piping washing is performed (s23). For piping washing, for example, a temperature of a printed board 22 is adjusted at around 25° C. with a temperature control system 14, a bypass piping 446 is purged with Milli Q water, and the air and Milli Q water are alternately introduced repeatedly, for example, for each 5 seconds. Then, a chip cartridge is washed (s24). For washing a chip cartridge, a drug solution introducing passway is switched from a bypass piping 446 to a piping 440, and the air and Milli Q water are alternately introduced into a piping 440 repeatedly, for example, for each 5 seconds. After it is confirmed by a liquid sensor 443 that a chip cartridge 11 has been filled with water, an introducing passway is switched to a bypass piping 446.

Then, purging in a piping is performed (s25). In purging in a piping, first, the air is introduced into a bypass piping 446 so as not to mix a buffer and Milli Q water. Then, the air and a buffer are alternately introduced into a bypass piping 446 repeatedly, for example, for each 5 seconds. It is confirmed by a liquid sensor 447 provided in a bypass piping 446 that a bypass piping 446 has been replaced with a buffer. Then, a buffer is injected into a chip cartridge (s26), in injection of a buffer into a chip cartridge, first, a bypass piping 446 is switched into a piping 440, the air and a buffer are alternately introduced into a chip cartridge 11 repeatedly, for example, for each 5 seconds. Then, a buffer is filled into a chip cartridge 11 (s27). In buffer filling, a buffer is introduced into a chip cartridge 11 while the state in a chip cartridge 11 is monitored with a liquid sensor 443, and an unnecessary sample is washed out by allowing to stand, for example, at 60° C. for 30 minutes (s28). After a step of washing out an unnecessary sample, a piping 440 is switched into a bypass piping 446, and washing in a piping is performed by introducing Milli Q water (s29). In this washing in piping, further, the air and Milli Q water are alternately introduced repeatedly, for example, for around each 5 seconds.

Then, washing in a chip cartridge is performed (s30). In washing in a chip cartridge, a bypass piping 446 is switched into a chip cartridge 11, and the air and water are alternately introduced repeatedly, for example, for around each 5 seconds. Thereafter, after it is confirmed by a liquid sensor 443 that Milli Q water has been filled into a chip cartridge 11, switching into a bypass piping 446 is performed. Then, measurement is initiated. In measurement, first, purging of a piping with an intercalator is performed (s31). In this purging of a piping with an intercalator, a waste is switched to an intercalator waste tank 464 while the air is introduced into a bypass piping 446. Then, the air and an intercalator are alternately supplied to a bypass piping 446 repeatedly, for example, for around each 5 seconds, and it is detected whether a bypass piping 446 has been replaced with an intercalator by using a liquid sensor 447.

Then, injection of an intercalator into a chip cartridge 11 is performed (s32). In this step, first, a bypass piping 446 is switched to a chip cartridge 11, and the air and an intercalator are alternately introduced repeatedly, for example, for around each 5 seconds. Then, under monitoring with a liquid sensor 443, an intercalator is filled into a chip cartridge 11 (s33). Thereafter, measurement is performed (s34). When measurement is complete, Milli Q water is introduced into a bypass piping 446, and then, the air and Milli Q water are alternately introduced, for example, for around each 5 seconds, and a piping is replaced with the air to perform washing in a piping (s35).

Finally, a bypass piping 446 is switched to a chip cartridge 11, the air and Milli Q water are alternately introduced, for example, for around each 5 seconds, and a chip cartridge 11 is further replaced with the air to perform washing in a chip cartridge (s36), thereby, a series of flowing steps are completed.

Like this, according to a step shown in FIG. 16 using a flow system 13 of FIG. 15, in order to effectively perform replacement of a drug solution, a sequence of flowing alternately the air and a drug solution in a piping is made like drug solution/air/drug solution/air, thereby, the solution can be flowed. By adopting such the flowing method, it is possible to minimize mixing of an old drug solution and a new drug solution in drug solution exchange. As a result, the transition state of liquid exchange is reduced, and final reproductivity of electrochemical property can be improved. Further, shortening in a flowing time and decrease in a drug solution amount can be realized by effective drug solution exchange. In addition, since a drug solution concentration in a reaction cell 115 can be usually retained constant by such the drug solution/air sequence flowing, in-plane uniformity of current property is improved, that is, reliability of detection is improved.

In addition, as a method of filling a drug solution into a cell 115, in the state where a two-way electromagnetic valve 444 as a chip cartridge outlet valve, after a pressure in a piping 440 on a chip cartridge downstream side is reduced (by controlling a two-way electromagnetic valve 451 in the state where a pump 454 is actuated, a pressure in a reduced pressure region 452 is reduced, and a two-way electromagnetic valve 453 is controlled to retain the reduced pressure state of a reduced pressure region 452), a two-way electromagnetic valve 44 is opened, thereby, a drug solution can be introduced into a chip cartridge reaction cell 115. In addition, timing of flowing shown in this FIG. 16 is only one example, and timing can be variously changed depending on an object, a subject and condition of measurement.

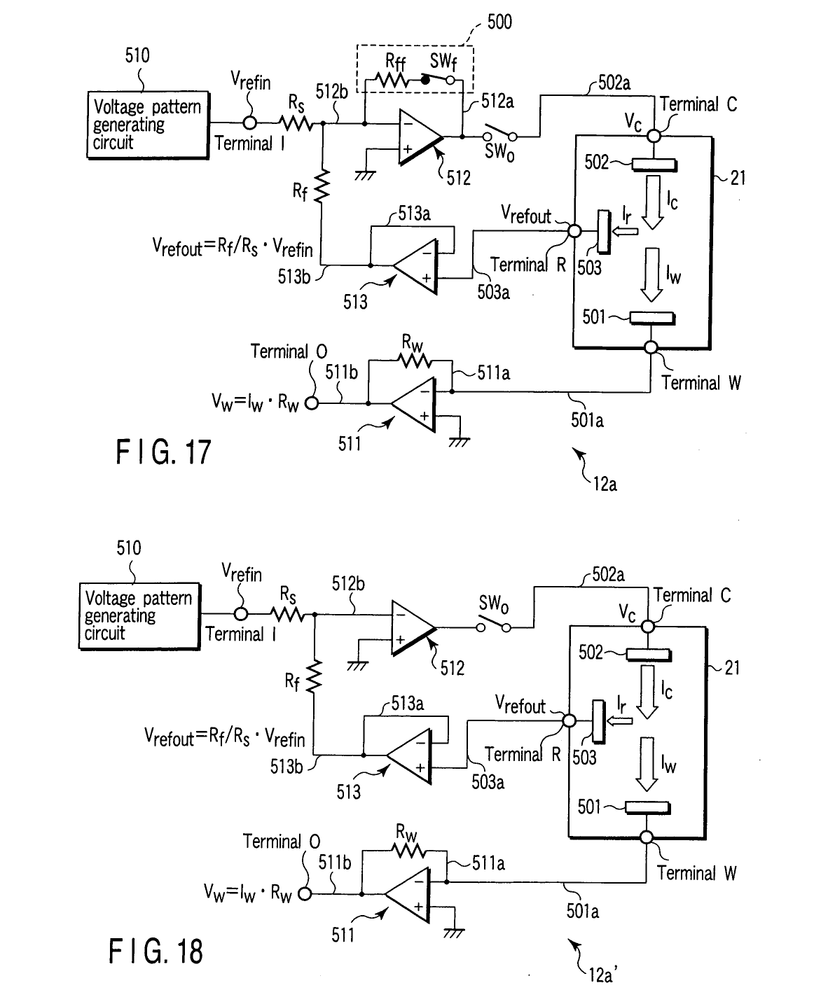

This FIG. 17 is a view showing a specific construction of a measuring system 12. A measuring system 12 shown in FIG. 17 is a 3 electrode-format potentiostat 12a for applying a desired voltage to a solution regardless of a variation in various conditions of an electrode or a solution in a cell 115, by feed-backing (negative feedback) a voltage of a reference electrode 503 relative to an input of a counter electrode 502. More specifically, a potentiostat 12a changes a voltage of a counter electrode 502 so that a voltage of a reference electrode 503 relative to a working electrode 501 is set at predetermined property, and measures an oxidation current of an intercalator electrochemically. A working electrode 501 is an electrode on which a nucleic acid probe having a target nucleic acid complementary with a target nucleic acid is immobilized, and is an electrode for detecting a reaction current in a cell 115. A counter electrode 502 is an electrode for applying a predetermined voltage between a working electrode 501 to supply a current into a cell 115. A reference electrode 503 is an electrode for feed-backing an electrode voltage to a counter electrode 502 so as to control a voltage between a reference electrode 503 and a working electrode 501 at predetermined voltage property, thereby, a voltage by a counter electrode 502 is controlled, and an oxidation current can be detected at a high precision while not influenced by various detection conditions in a cell 115. A voltage pattern generating circuit 510 generating a voltage pattern for detecting a current flowing between electrodes is connected to a inversion input terminal of a inversion amplifier 512 (OPc) for controlling a reference voltage of a reference electrode 503 via a wiring 512b.

A voltage pattern generating circuit 510 is a circuit for converting a digital signal input from a control mechanism 15 into an analogue signal to generate a voltage pattern, and is provided with a DA converter. A resistor Rs is connected to a wiring 512b. A non-inversion input terminal of an inversion amplifier 512 is grounded, and a wiring 502a is connected to an output terminal. A wiring 512b on an inversion input terminal side of an inversion amplifier 512 and a wiring 502a on an output terminal side are connected with a wiring 512a. In this wiring 512a, a protecting circuit 500 consisting of a feedback resistor Rff and a switch SWf is provided. A wiring 502a is connected to a terminal C. A terminal C is connected to a counter electrode 502 on a chip 21. When a plurality of counter electrodes 502 are provided, terminals C are connected parallel to respective electrodes. Thereby, by one voltage pattern, a voltage can be applied to a plurality of counter electrodes 502 at the same time. In a wiring 502a, a switch SWo for controlling on-off of application of a voltage to a terminal C is provided.

A protecting circuit 500 provided in an inversion amplifier 512 is configured not to apply an excessive voltage to a counter electrode 502. Therefore, an excessive voltage is applied at measurement, and a solution is electrolyzed, thereby, stable measurement becomes possible without influencing on detection of an oxidation current of a desired intercalator. A terminal R is connected to a non-inversion input terminal of a voltage follower amplifier 513 (OPr). An inversion input terminal of a voltage follower amplifier is short-circuited with a wiring 513b and a wiring 513a connected to its input terminal. In a wiring 513b, a resistor Rf is provided, and this is connected between a resistor of a wiring 512b, and an intersection of a wiring 512a and a wiring 512b. Thereby, a voltage obtained by feed-backing a voltage of a reference electrode 503 to a voltage pattern generated by a voltage pattern generating circuit 510 is inputted in an inversion amplifier 512, and based on an output obtained by inversion-amplifying such the voltage, a voltage of a counter electrode 502 is controlled.

A terminal W is connected to an inversion input terminal of a trans impedance amplifier 511 (OPw) with a wiring 501a. A non-inversion input terminal of a trans impedance amplifier 511 is grounded, a wiring 511b connected to its terminal, and a wiring 501a are connected with a wiring 511a. In a wiring 511a, a resistor Rw is provided. Letting a voltage at an terminal O on an output side of this trans impedance amplifier 511 to be Vw, and a current to be Iw, then, Vw=Iw·Rw. An electrochemical signal obtained from this terminal O is outputted in a control mechanism 15. Since there are plural working electrodes 501, a plurality of terminals W and terminals O are provided corresponding to respective working electrodes 501. Outputs from a plurality of terminals O are switched with a signal switching part described later, and subjected to analog to digital conversion, thereby, an electrochemical signal from each working electrode 501 can be obtained as a digital value almost at the same time. Alternatively, a circuit such as a trans impedance amplifier 511 between a terminal W and a terminal O may be shared by a plurality of working electrodes 501. In this case, a wiring 501a may be provided with a signal switching part for switching a wiring 501a from a plurality of terminals W.

The effect of a measuring system 12 using this potentiostat 12a of FIG. 17 will be explained by comparing the case of use of the previous potentiostat. The previous potentiostat is shown in FIG. 18. As shown in FIG. 18, a construction of the previous potentiostat 12a′ is approximately common with that of a potentiostat 12a shown in FIG. 17. A difference is in that a protecting circuit 500 is not provided in an inversion amplifier 512. A voltage at an output terminal I of a voltage pattern generating circuit 510 is Vrefin, a voltage at a terminal C is Vc, and a voltage at a terminal R is Vrefout. Feedback of a reference electrode 503 leads to Vrefout=Rf/Rs·Vrefin.

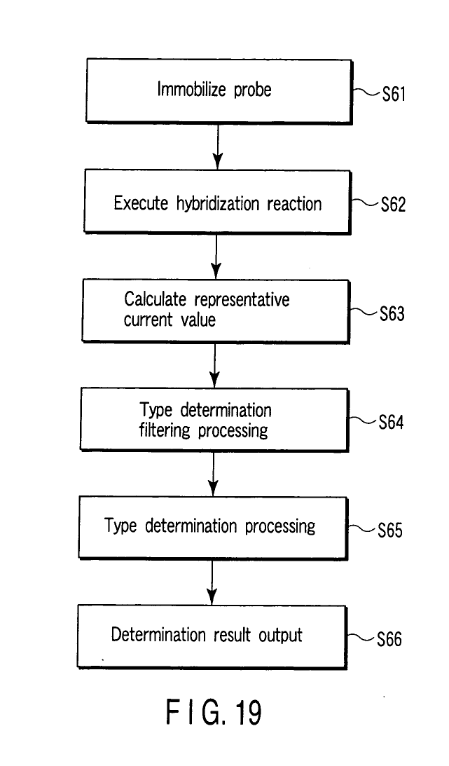

Then, one example of a measurement data analyzing procedure for performing signal analysis with a computer 16 based on measurement data will be explained. Herein, an analysis procedure of genotype determination of determining whether a nucleotide at a SNP position of a target nucleic acid is G type (homo type), T type (homo type) or GT type (hetero type) will be explained using a flowchart of FIG. 19. Although not expressly indicated in FIG. 5, a main processor 16a of a computer 16 executes type determination filtering, type determination treatment, and determination result outputting by executing an analysis program consisting of a plurality of commands for performing genotype determination filtering, genotype determination treatment, and determination result outputting. In addition, for controlling the aforementioned control mechanism 15, a control program is provided separately. These analysis program and control program may be executed by reading out of an analysis program stored in a recording medium by a recording medium reading device provided in a computer 16, or may be executed by reading out from a memory device such as a magnetic disk provided in a computer 16.

As assumption for performing this measurement data analysis, an example of the 4-nucleotide substitution type SNP will be explained. First, four kinds of A, G, C and T of nucleotides at a SNP position as a target nucleic acid to be detected are prepared, plural (per kind) nucleic acid probes having nucleotide sequences complementary to the target nucleic acid are immobilized on each working electrode 501. Separately, a plurality of nucleic acid probes (hereinafter, referred to as negative control) having nucleotide sequences different from these four kinds of nucleic acid probes are immobilized on other working electrode 501 (s61). A kind of a nucleic acid probe immobilized on one working electrode 501 is one, in principle.

Then, a sample containing a specimen target nucleic acid is injected in a chip on which the aforementioned nucleic acid probe is immobilized, to generate a hybridization reaction (s62), and via washing with a buffer, and an electrochemical reaction by introduction of an intercalator, a representative current value is calculated using a measurement system 12 (s63). A representative current value refers to a numerical value effective for quantitatively grasping occurrence of a hybridization reaction of each nucleic acid probe, and one example is a maximum of a current value of a signal to be directed (peak current value). A peak current value is obtained by measuring an oxidation current signal from an intercalator bound to a double-stranded nucleic acid hybridized with a nucleic acid probe immobilized on each working electrode 501, and obtaining a peak of the current value. It is desirable to perform detection of a peak current value by subtracting a background current other than an oxidation current signal from an intercalator. Of course, any value may be adopted as a representative current value depending on a precision and an object of signal treatment, and an example is an integrated value of an oxidation current signal. Of course, an example is not limited to a current value, but a voltage value, or a value obtained by performing numerical value analysis treatment on a current and a voltage may be adopted as a representative value.

Measurement data regarding a target nucleic acid where nucleotides at a SNP position are A, G, C and T types, that is, representative current values are defined as Xa, Xg, Xc and Xt, respectively, and a representative current value of a nucleic acid probe of a negative control is defined as Xn. In addition, since a plurality of representative current values are obtained depending on respective kinds, first Xa is defined as Xa1, second Xa is defined as Xa2, and so on in order to discriminate them from each other. In addition, the number of resulting representative current values of a target nucleic acid where nucleotides at a SNP position are A, G, C and T types is defined as na, ng, nc and nt, and the number of a representative current value obtained regarding a negative control is defined as nn.

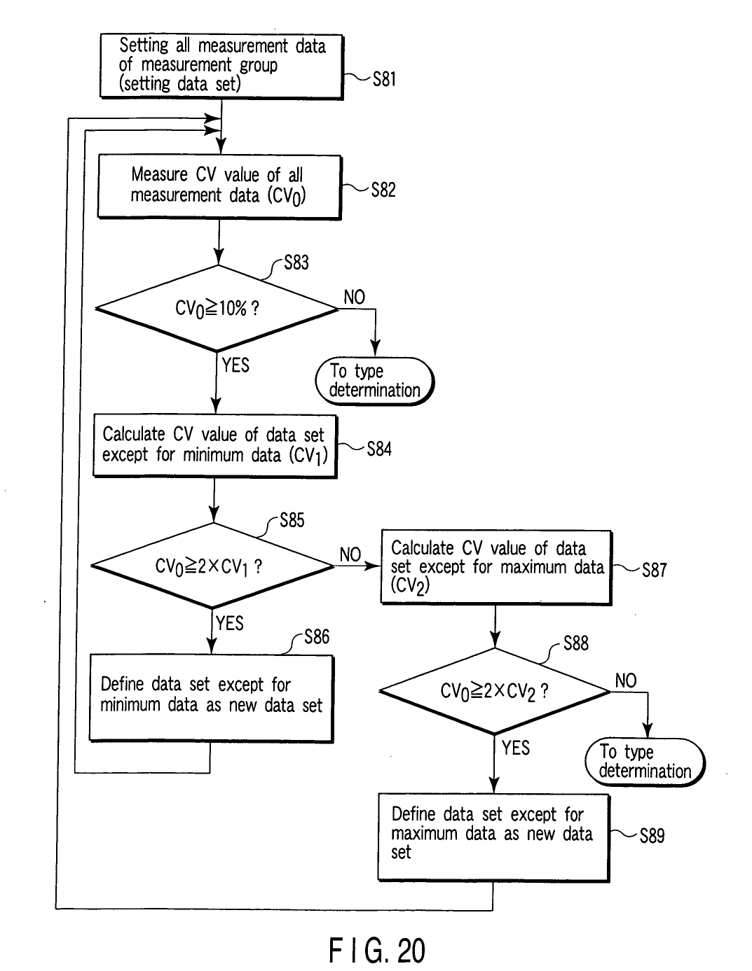

Then, in order to exclude clearly abnormal data among resulting representative current values Xa, Xg, Xc, Xt and Xn, type determination filtering treatment is executed (s64). A flowchart of this type determination filtering treatment is shown in FIG. 20. This type determination filtering treatment of FIG. 20 is performed separately regarding Xa, Xg, Xc, Xt and Xn, respectively. For example, in the case of an example of Xa, among na representative current values obtained for Xa, representative current values which seem to be clearly abnormal data are excluded by this type determination filtering. Regarding Xg, Xc, Xt and Xn, the similar procedure is performed. In addition, in explanation of FIG. 20, since the similar processing is performed depending on a kind of data, an example of filtering of Xa will be explained. Specifically, as shown in FIG. 20, first, all measurement data of a measurement group is set, that is, a data set is set (s81). For example, in the case of Xa, Xa1, Xa2, . . . Xana are set as a data set.

Then, regarding these measurement data Xa1, Xa2, . . . Xana, a CV value (hereinafter, CV0) is calculated (s82). This CV0 is obtained by diving a standard deviation of measurement data Xa1, Xa2, . . . Xana by an average value. It is determined whether the resulting value CV0 is 10% or more, that is, 0.1 or more (s83). If 10% or more, a CV value of na-1 data set obtained by removing a minimum from measurement data (hereinafter, CV1) is calculated (s84). If less than 10%, it is determined that there is no clearly abnormal data, and the procedure progresses to type determination described later. After calculation of CV1, it is determined whether CV0≧2×CV1 is established or not (s85). If this inequality is established, a procedure goes to (s86), na-2 data set obtained by removing a minimum from measurement data is further defined as a data set newly, a procedure is returned to (s82), and abnormal data filtering is repeated. If an inequality is not established, it is determined that abnormal data is present not on a minimum side but on maximum side, and a CV value of na-2 data set obtained by removing a maximum from measurement data (hereinafter, CV2) is calculated (S87). It is determined whether CV0≧2×CV2 is established or not (S88). If established, na-3 data set obtained by removing a maximum from measurement data is further defined as a data set newly, a procedure is returned to (s82), and abnormal data filtering is repeated. If not established, it is determined that there is no clearly abnormal data, and a procedure goes to type determination described later. The aforementioned type determination filtering is performed also regarding Xg, Xc, Xt and Xn.

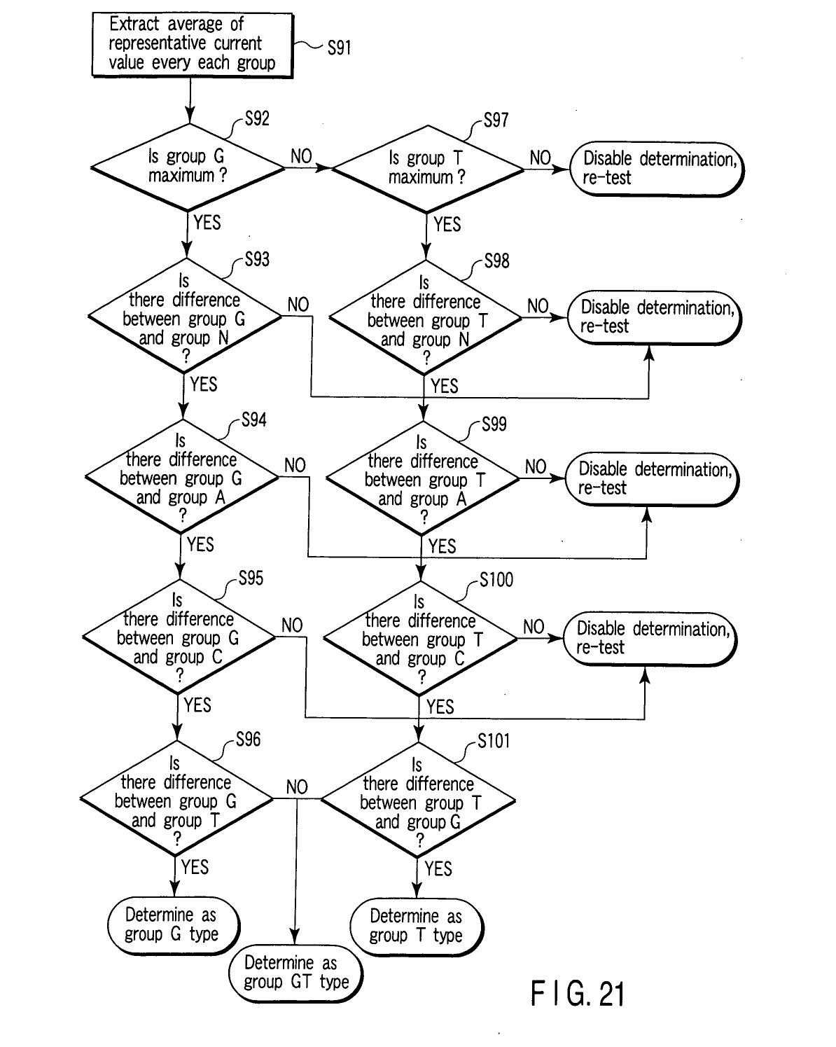

Then, type determination treatment is executed by use of the resultant type determination filtering result (s65). One example of this type determination treatment will be explained using a flowchart of FIG. 21. In addition, an example of FIG. 21 indicates the case of type determination determining whether a nucleotide at a SNP position of a target nucleic acid is G type, T type or GT type. This type determination treatment roughly consists of maximum group determining algorithm, and 2 sample t-test algorithm. As shown in FIG. 21, first, an average of a representative current value every each group is extracted (s91). A group includes Xa, Xg, Xc, Xt and Xn, a different target nucleic acid is different group, and the same target nucleic acid is the same group. Measurement data from which clearly abnormal data has been excluded by type determination filtering in (s64) is extracted. Of course, measurement data from which abnormal data has been excluded by filtering other than type determination filtering of (s64) may be extracted, or measurement data without any filtering may be extracted. In addition, not an average of a representative current value, but another statistically processed value obtained by statistic treatment from these statistical values may be obtained. The case where nucleotides at a SNP position of a target nucleic acid are A, G, C and T are groups A to T, and a negative control is a group N, and this case will be explained. In addition, resulting average values Xa, Xg, Xc, Xt and Xn are Ma, Mg, Mc, Mt and Mn regarding respective groups.

Then, regarding the resulting averages Ma, Mg, Mc, Mt and Mn, it is determined whether a maximum is an average Mg of a group G or not (s92). If a maximum, a procedure goes to (s93), and if not a maximum, a procedure goes to (s97). In (s97), regarding averages Ma, Mg, Mc, Mt and Mn, it is determined whether a maximum is an average Mt of a group T or not. If a maximum, a procedure goes to (s98), and if not a maximum, which results in that groups G and T are both not a maximum and a test is performed again due to disable determination. In (s93), it is determined whether there is a difference between group G measurement data Xg1, Xg2, . . . and group N measurement data Xn1, Xn2, . . . . For determining whether there is a difference or not, for example, 2 sample T-test is used. Specifically, a representative relationship between a probability P and a significance level ax obtained by a 2 sample T-test is compared, and this is determined as follows:

H0: if P≧α, there is not significant difference (null hypothesis)

H1: if P<α, there is significant difference (conflict hypothesis).

A significance level ax can be set by a user using a computer 16. In this example of (s93), a question H1 if there is a difference in a value between measurement data of a group G and measurement data of a group N is proposed, and against this question, hypothesis H0 postulating that there is no difference between these two groups is set. Provided that a difference between two groups are summarized in an average Mg of measurement data of a group G and an average Mn of measurement data of a group N, a probability is obtained. For calculating a probability, based on statistic values Xg1, Xg2, . . . of a group G, and statistic values Xn1, Xn2, . . . of a group N, a statistic constant t and a freedom degree φ are calculated, and a probability P is obtained from an integrated value of a probability density variable of a t distribution. Regarding the resulting probability P, if P≧α, H0 cannot be rejected, and determination is reserved. That is, it is determined that there is no difference. If P<α, H0 is rejected, and hypothesis H1 is adopted, and it is determined that there is a difference. When determination result is determined that “there is a difference” in this way, a procedure goes to (s94), and when it is determined “there is no difference”, a test is performed again due to disable determination.

In (s94), regarding a group G and a group A, it is determined whether there is a difference between two groups or not by using a 2 sample t-test as in (s93). If there is a difference, a procedure goes to (s95), and if there is no difference, a test is performed again due to disable determination. In (s95), regarding a group G and a group C, it is determined whether there is a difference between two groups or not by using the same 2 sample t-test as that of (s93). If there is a difference, a procedure goes to (s96), and if there is no difference, a test is performed again due to disable determination. In (s96), regarding a group G and a group T, it is determined whether there is a difference between two groups or not by using the same 2 sample t-test as that of (s93). If there is a difference, this is determined to be a group G type, because a group G type is a maximum average, and there is a difference between other measurement group. If there is no difference, this is determined to be a group GT type, because a group G type is a maximum average, but there is no difference in measurement result between a group G type and a group T type. In (s98), regarding a group T and a group N, it is determined whether there is a difference between two groups by using the same 2 sample t-test as that of (s93). If there is a difference, a procedure goes to (s99), and if there is not difference, a test is performed again due to disable determination. In (s99), regarding a group T and a group A, it is determined whether there is a difference between two groups or not by using the same 2 sample t-test as that of (s93). If there is a difference, a procedure goes to (s100), and if there is not difference, a test is performed again due to disable determination. In (s100), regarding a group T and a group C, it is determined whether there is a difference between two groups or not by using the same 2 sample t-test as that of (s93). If there is a difference, a group goes to (s101), and there is not a difference, a test is performed again due to disable determination. In (s101), regarding a group T and a group G, it is determined whether there is a difference between two groups or not by using the same 2 sample t-test as that of (s93). If there is a difference, this is determined to be a group T type, because a group T type is a maximum average, and there is a difference between other measurement groups. If there is no difference, this is determined to be a group GT type, because a group T type is a maximum average, but there is no difference in measurement result between a group T type and a group G type.

The above determination results are displayed in a not shown display device provided in a computer 16 (s66). By using such the type determination algorithm, it becomes possible to determine a hetero type.

Although in FIGS. 19 to 21, a procedure for determining whether a type corresponds to any of G type, T type and GT type was shown, the procedure can be of course applied to determination of any two type of A type, G type, a C type and T type, or hetero of them. In addition, it is not necessarily required that measurement data is obtained regarding four kinds of A type, G type, C type and T type groups, or data may be obtained regarding only two groups with respect to possible to nucleotides of SNP, or one group of a negative control may be added to those two groups.

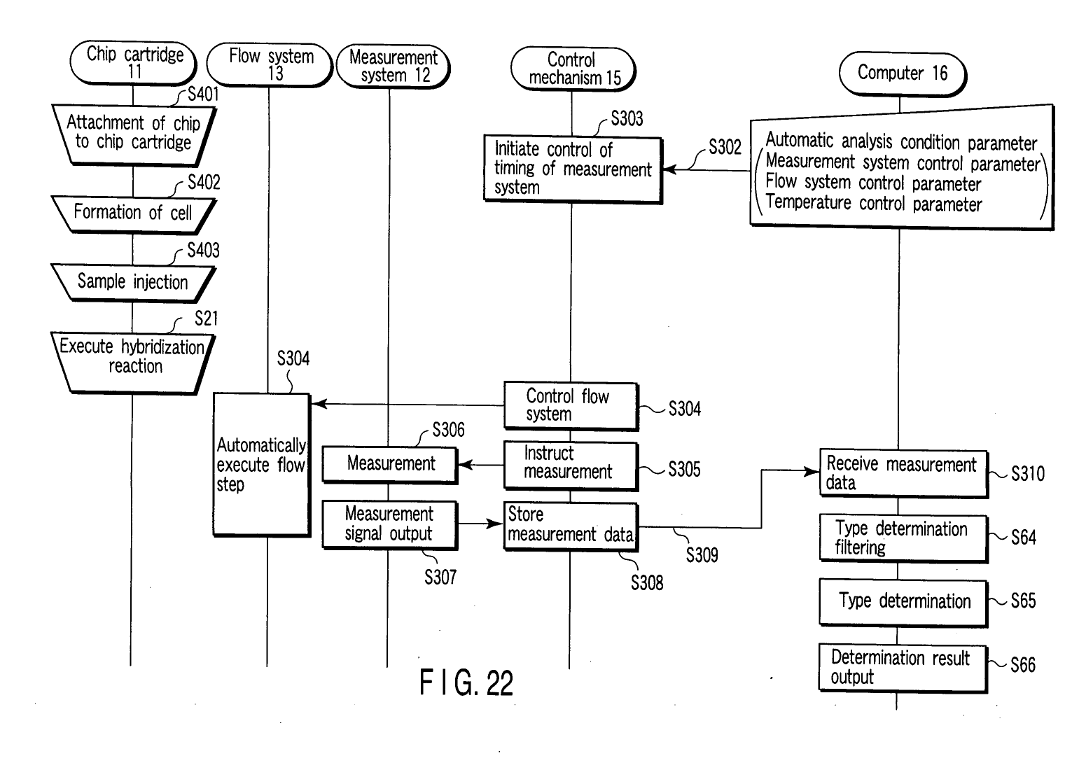

An automatic analyzing procedure for individual discrimination using the aforementioned individual discriminating test apparatus will be explained using a sequence view of FIG. 22. As shown in FIG. 22, first, using a computer 16, automatic analyzing condition parameters for automatic analysis are set, and a user instructs a computer 16 to execute automatic analysis based on set automatic analysis condition parameters (s301). An automatic analysis condition parameter is a control parameter for controlling a control mechanism 15. A control parameter used in a control mechanism 15 consists of a measurement system control parameter for controlling a measurement system 12, a flow system control parameter for controlling a flow system 13, and a temperature control system control parameter for controlling a temperature control system 14. A measurement system control parameter is an input setting parameter, and consists of an initial value, an inclement value, a completion value, a measurement time interval, and a motion mode.

A flow system control parameter has an electromagnetic control parameter for controlling electromagnetic valves 403, 413, 423, 433, 441, 442, 444, 445, 451, 453 and 463 shown in FIG. 15, a sensor control parameter for controlling liquid sensors 443 and 447, and a pump control parameter for controlling a pump 454. These electromagnetic valve controlling parameter, sensor control parameter and pump control parameter include a control amount of a control subject, control timing of a control subject, and control condition for controlling a control subject as details of a parameter, as condition for executing a series of steps as shown in (s22) to (s36) of FIG. 16 sequentially.

A temperature control parameter is given, in principle, accompanying with a flow system control parameter. That is, by setting a flow system control parameter, a temperature control parameter is set corresponding to motion of a flow system 13. Thereby, it becomes possible to control a temperature of a temperature control system 14 in conjunction with a flow system 13.

By execution of automatic analysis, an automatic analysis condition parameter is sent to a control mechanism 15 (s302). Among a received automatic analysis condition parameter, based on a measurement system control parameter, a control mechanism 15 controls a measurement system 12, and based on a flow system control parameter, the mechanism controls a flow system 13, and based on a temperature control system control parameter, the mechanism controls a temperature control system 14. In addition, a control mechanism 15 manages timing for controlling these measurement system 12, flow system 13, and temperature controlling system 14 based on control timing and control condition containing in each control parameter. Therefore, a control sequence can be freely determined by an automatic analysis condition parameter set by a user, but in this FIG. 22, a representative one example will be explained.

Separately from this automatic analysis, a user prepares a chip cartridge 11. Thereupon, first, a printed board 22 on which an individual discrimination chip 21 is sealed, in which a desired nucleic acid probe is immobilized on a working electrode 501, is immobilized on a support 111 of a chip cartridge 11 with a substrate fixing screw 25, thereby, attachment to a chip cartridge 11 is performed (s401). A chip cartridge upper lid 112 integrated with a sealing material 24a with an upper lid fixing screw 117 and a support 111 are fixed, and this is prepared in the state where a cell 115 is formed (s402). A sample is injected into a chip cartridge 11 through an injection port 119 (s403). A chip cartridge 11 is mounted on an apparatus body, and an initiation procedure is performed, thereby, a hybridization reaction (s21) is initiated. It is desirable that a volume of a sample to be injected is slightly larger than an amount of a volume of a cell 115. Thereby, a cell 115 can be completely filled with a sample, leaving no air.

A control mechanism 15 initiates control of timing of a measurement system based on a measurement system control parameter received from a computer 16 (s303). In addition, a control mechanism 15 successively controls each element of a flow system 13 based on a flow system control parameter received from a computer 16 (s304). In addition, although not particularly shown in FIG. 22, in conjunction with control of this flow system 13, a temperature of a temperature control system 14 is controlled based on a temperature control system control parameter. By this control, a flow system 13 automatically performs a flowing step including a hybridization reaction shown in (s21) to (s36) (except for s34) of FIG. 16 (s305), and at the same time, a temperature control system 14 is automatically controlled so that an individual discrimination chip 21 is set at a temperature designated in the flowing step. A control mechanism 15 instructs a measurement system 12 to perform measurement synchronously with timing of a measurement step midway of this flowing step (s34) (s305). That is, at timing of a measurement step of a flowing step (s34), an initial value, an inclement value, a completion value, a measurement time interval, and a motion setting mode are stored in an initial value register 151, an inclement value register 152, a completion value register 153, an interval register 154 and a motion setting register 155 of a control mechanism 15. Alternatively, measurement system timing control of the (s303) may be performed at the same time with this (s305).

A measurement system 12, based on this measurement instruction, performs measurement, for example, by generating a voltage pattern (s306), and the resulting measurement signal is input in a control mechanism 15 from a terminal O (s307). A control mechanism 15 processes a received measurement signal, and stores this as measurement data in a data memory 15b (s308). This measurement data is input in a computer 16 via a local bus 17 (s309). A computer 16 receives this measurement data (s310).

When necessary measurement data is obtained in this way, a computer 16 executes type determination filtering shown in FIG. 20 (s64) based on measurement data. When type determination filtering is complete, based on filtered data, type determination treatment shown in FIG. 21 is executed (s65). The resulting determination treatment result is displayed on a display device equipped on a computer 16 (s66).

The above type determination is performed regarding each of an individual to be discriminated, and a sample, and data of results is stored in a computer 16. A computer 16 compares those results, and determines whether they are consistent or not. In addition, reliability of determination result is determined from pre-inputted data of an allele frequency and a genotype frequency, and this may be displayed with the determination result.

Allotment of processing between a computer 16 and a control mechanism 15 is not limited to the aforementioned allotment. For example, when a measurement system 12, a flow system 13, and a temperature control system 14 have a processor for interpreting an instruction from a computer 16 and executing each element, a control mechanism 15 may be omitted.

For managing timing of a measurement system 12, a flow system 13 and a temperature control system 14, when these measurement system 12, flow system 13 and temperature control system 14 have a processor for managing timing, each processing is executed based on timing managed by the processor. In this case, if a computer 16 sends an automatic analysis condition parameter to these measurement system 12, flow system 13 and temperature control system 14, it is not necessary to manage timing. Alternatively, a computer 16 may perform control of timing of a measurement system 12, a flow system 13, a temperature control system 14 and a control mechanism 15.

In addition, an example of an injection port 119 communicating with an outlet port 116b has been shown, but the port may be communicated with an inlet port 116a. In addition, a working electrode 501 and a bonding pad 221 on an individual discrimination chip 21 have been shown as a laminated structure of Ti or Au, but an electrode and a pad using other material may be used. In addition, arrangement of a working electrode 501 is not limited to that shown in FIG. 14. The number of electrodes of each of a working electrode 501, a counter electrode 502 and a reference electrode 503 is not limited to that shown in the figures.

In addition, a flow system 13 is not limited to that shown in FIG. 15. For example, depending on a kind of a reaction, by adding a supply system for supplying a drug solution other than the air, Milli Q water, a buffer and an intercalator, or a gas, a more complicated reaction may be performed in a cell 115. In addition, control of a supply passway and a supply amount for a drug solution between pipings may be performed using a means other than an electromagnetic valve. A motion of a flow system 13 shown in FIG. 16 may be only one example, and a motion may be variously changed depending on an object of a reaction.

In addition, flow channels 601a to 601d are not limited to arrangement shown in FIG. 9B. For example, a detection flow channel 601a may be disposed parallel with a straight line connecting cell pore parts 115a and 115b, or respective flow channels 601a to 601d may not be straight flow channels, but may be curved flow channels. Further, an example in which an inlet port 116a and an outlet port 116b extend vertical to a cell bottom surface has been shown, being not limited thereto, and ports may be configured to extend parallel with a cell bottom surface.

According to the embodiment of the present invention as described above, an individual discriminating test and determination of the result can be performed automatically.

### EXAMPLE

Table 1 shows an example of selected single nucleotide polymorphisms. For each single nucleotide polymorphism, an allele frequency of each nucleotide and a genotype frequency in the case of Asian (Chinese or Japanese) as a sample are described.

Table 2 shows estimated heterozygosity calculated from a genotype frequency of Table 1, and a maximum frequency (Max value) and a minimum frequency (Minimum value) in each genotype (C/C, T/T, C/T), and discriminating ability (power of identification) expressed by a reciprocal thereof (one person per how many persons). A lowest step of each column of discriminating ability is obtained by multiplying discriminating ability, respectively. Therefore, regarding 22 SNPs in Table 1, it is seen that, in even a highest frequency combination, one person per about 8.38×10⁶ persons (about 8380 thousands persons) has the same genotype, and in a lowest frequency combination, one person per 5.22×10¹⁹ persons has the same genotype.

TABLE 1

| | | Allele | Allele | Genotype | Genotype |
|---|---|---|---|---|---|
| dbSNP | Chromosome | frequency | frequency | frequency | frequency |
| number | number | (C) | (T) | (C/C) | (T/T) |
| rs734664 | 1 | 0.28 | 0.72 | 0.0784 | 0.5184 |
| rs772436 | 2 | 0.72 | 0.28 | 0.5184 | 0.0784 |
| rs716360 | 4 | 0.73 | 0.27 | 0.5329 | 0.0729 |
| rs730907 | 5 | 0.56 | 0.44 | 0.3136 | 0.1936 |
| rs927628 | 6 | 0.46 | 0.54 | 0.2116 | 0.2916 |
| rs997556 | 7 | 0.56 | 0.44 | 0.3136 | 0.1936 |
| rs919023 | 8 | 0.51 | 0.49 | 0.2601 | 0.2401 |
| rs997750 | 10 | 0.6 | 0.4 | 0.36 | 0.16 |
| rs959566 | 12 | 0.38 | 0.62 | 0.1444 | 0.3844 |
| rs1105576 | 13 | 0.63 | 0.37 | 0.3969 | 0.1369 |
| rs911621 | 14 | 0.33 | 0.67 | 0.1089 | 0.4489 |
| rs877228 | 15 | 0.48 | 0.52 | 0.2304 | 0.2704 |
| rs727206 | 17 | 0.66 | 0.34 | 0.4356 | 0.1156 |
| rs1017415 | 18 | 0.66 | 0.34 | 0.4356 | 0.1156 |
| rs1000329 | 19 | 0.32 | 0.68 | 0.1024 | 0.4624 |
| rs743018 | 20 | 0.68 | 0.32 | 0.4624 | 0.1024 |
| rs18579 | 21 | 0.61 | 0.39 | 0.3721 | 0.1521 |
| rs738518 | 22 | 0.45 | 0.55 | 0.2025 | 0.3025 |
| rs715262 | 16 | 0.3 | 0.7 | 0.09 | 0.49 |
| rs868454 | 9 | 0.3 | 0.7 | 0.09 | 0.49 |
| rs1027185 | 3 | 0.3 | 0.7 | 0.09 | 0.49 |
| rs179479 | 11 | 0.3 | 0.7 | 0.09 | 0.49 |

TABLE 2

| | | | | Power of | Power of |
|---|---|---|---|---|---|
| | | Max value | Minimum | identification | identification |
| dbSNP | Estimated | in the | value in the | for the | for the |
| number | heterozygosity | 3 types | 3 types | Max value | Minimum value |
| rs734664 | 0.4032 | 0.5184 | 0.0784 | 1.929 | 12.755 |
| rs772436 | 0.4032 | 0.5184 | 0.0784 | 1.929 | 12.755 |
| rs716360 | 0.3942 | 0.5329 | 0.0729 | 1.877 | 13.717 |
| rs730907 | 0.4928 | 0.4928 | 0.1936 | 2.029 | 5.165 |
| rs927628 | 0.4968 | 0.4968 | 0.2116 | 2.013 | 4.726 |
| rs997556 | 0.4928 | 0.4928 | 0.1936 | 2.029 | 5.165 |
| rs919023 | 0.4998 | 0.4998 | 0.2401 | 2.001 | 4.165 |
| rs997750 | 0.48 | 0.48 | 0.16 | 2.083 | 6.250 |
| rs959566 | 0.4712 | 0.4712 | 0.1444 | 2.122 | 6.925 |
| rs1105576 | 0.4662 | 0.4662 | 0.1369 | 2.145 | 7.305 |
| rs911621 | 0.4422 | 0.4489 | 0.1089 | 2.228 | 9.183 |
| rs877228 | 0.4992 | 0.4992 | 0.2304 | 2.003 | 4.340 |
| rs727206 | 0.4488 | 0.4488 | 0.1156 | 2.228 | 8.651 |
| rs1017415 | 0.4488 | 0.4488 | 0.1156 | 2.228 | 8.651 |
| rs1000329 | 0.4352 | 0.4624 | 0.1024 | 2.163 | 9.766 |
| rs743018 | 0.4352 | 0.4624 | 0.1024 | 2.163 | 9.766 |
| rs18579 | 0.4758 | 0.4758 | 0.1521 | 2.102 | 6.575 |
| rs738518 | 0.495 | 0.495 | 0.2025 | 2.020 | 4.938 |
| rs715262 | 0.42 | 0.49 | 0.09 | 2.041 | 11.111 |
| rs868454 | 0.42 | 0.49 | 0.09 | 2.041 | 11.111 |
| rs1027185 | 0.42 | 0.49 | 0.09 | 2.041 | 11.111 |
| rs179479 | 0.42 | 0.49 | 0.09 | 2.041 | 11.111 |
| | | | | 8.38E+06 | 5.22E+19 |

In Detailed Explanation of the present invention, as a method of calculating a probability that a combination of genotypes is present, there was described that frequencies of genotypes are multiplied, and thereafter, a reciprocal is taken. However, as in the present Example, a reciprocal for a genotype is pre-calculated as discriminating ability regarding each SNP, and thereafter, multiplication may be performed.

Then, among SNPs shown in the above Table, arbitrary five SNPs were selected, and an array on which nucleic acid probes for detecting the SNPs were immobilized was manufactured. Sequences of nucleic acid probes used are shown in Table 3.

TABLE 3

Electrode

| | | |
|---|---|---|
| No. | SNP-base | Sequence (5′-3′) |

| | | |
|---|---|---|
| 1-3 | rs734664-G | tcttccgtcctgcttt-SH |
| 4-6 | rs734664-A | tcttccatcctgctttt-SH |
| 7-9 | rs772436-G | gtggaatcggaaaagag-SH |
| 10-12 | rs772436-A | gtggaatcagaaaagagt-SH |
| 13-15 | rs716360-A | ccaagtgcactctatgg-SH |
| 16-18 | rs716360-G | ccaagtgcgctctatg-SH |
| 19-21 | rs730907-G | aggagtagggagcagc-SH |
| 22-24 | rs730907-A | aggagtagagagcagc-SH |
| 25-27 | rs927628-G | tgggcacgtcagtat-SH |
| 28-30 | rs927628-A | tgggcacatcagtatt-SH |

-SH; thiol modification

A solution containing each nucleic acid probe (30 μg/mL) and NaCl (400 mM) was spotted on electrodes of an array, and this was allowed to stand for 1 hour. Thereafter, this was washed with distilled water, and air-dried to obtain a nucleic acid-immobilized chip.

As a target nucleic acid, a region which is amplified by PCR was synthesized, and this was used as a model sample. Table 4 shows sequences of synthetic oligos used as a target nucleic acid.

TABLE 4
Target nucleic acid (synthetic oligo)

rs734664-G
GTTTTTGCCTAAAAGCAGGACGGAAGAAGGGAAGGAAAAAGGGAAGGGAA
TGAAAAAGGCCAGGGGAGGGCTGGGGAGGGAAGCGAAGGGAGAAGACTCT
rs734664-A
GTTTTTGCCTAAAAGCAGGATGGAAGAAGGGAAGGAAAAAGGGAAGGGAA
TGAAAAAGGGGAGGGGAGGGCTGGGGAGGGAAGGGAAGGGAGAAGACTCT
rs772436-G
TTTCTTATATTACTCTTTTCCGATTCCACTTTCCAAAATAAGTGACCTGC
ATCCACACCCTGCCTGAAGCTCTGCTTTTTGGGCTCTACTGGCTAAGACA
rs772436-A
TTTCTTATATTACTCTTTTCTGATTCCACTTTCCAAAATAAGTGACCTGC
ATCCACACCCTGCCTGAAGCTCTGCTTTTTGGGCTCTACTCGCTAAGACA
rs716360-A
TCTCTTTCTTCCCATAGAGTGCACTTGGGGATTGTCTACCATAATAAATG
ACCGGTCTTTCAAATGAATGGTTTATCCACTTTGATCCTGGTATGTCCAA
rs716360-G
TCTCTTTCTTCCCATAGAGCCCACTTCGCGATTGTCTACCATAATAAATC
AGCGCTCTTTCAAATGAATCGTTTATCCACTTTGATCCTCGTATGTCCAA
rs730907-G
TTAGAGTGAAAGGCTGCTCCCTACTCCTTTACATGTATCCACCTTGGGAG
ACTTATTTCTTTTACTTATGGTCATCTCTTGTGTCCTTCAAAAGCTACAA
rs730907-A
TTACAGTGAAAGGCTGCTCTCTACTCCTTTACATGTATCCACCTTGGGAG
ACTTATTTCTTTTACTTATCGTCATCTCTTGTGTCCTTCAAAAGGTACAA
rs927628-G
AATTGCTTTTGAATACTGACGTGCCCAAAGTTAAAAAACTATAAATGGGT
CCTTGGTCAGCTAATTCCAAAACAATACACCCCAGACTTCTGTTAACACT
rs927628-A
AATTGCTTTTGAATACTGATGTGCCCAAAGTTAAAAAACTATAAATGGCT
CCTTGGTCAGCTAATTCCAAAACAATACACCCCAGACTTCTGTTAACACT

Each synthetic nucleic acid shown in Table 4 was dissolved in a 2×SSC solution to 1×10¹⁴ copy/mL, to obtain a target nucleic acid solution. Among synthetic nucleic acids shown in Table 4, a nucleic acid containing G at a polymorphism site was prepared as a sample 1, and a nucleic acid containing A at a polymorphism site was prepared as a sample 2. A manufactured array was immersed in 50 μL of a prepared target nucleic acid solution, to hybridize a nucleic acid probe on an array and a target nucleic acid in a target nucleic acid solution. The array was immersed in a 0.2×SSC solution (35° C.) for 40 minutes to wash away a non-specifically bound target nucleic acid. This was washed with ultrapure water, and air-dried. Hoechst 33258 (50 μM) was dissolved in a 20 mM phosphate buffer (100 mM NaCl), the air-dried chip was immersed in this solution, and after 5 minutes, electrochemical measurement was performed. A height of an oxidation peak of Hoechst 33258 was detected, and this was adopted as a peak current value.

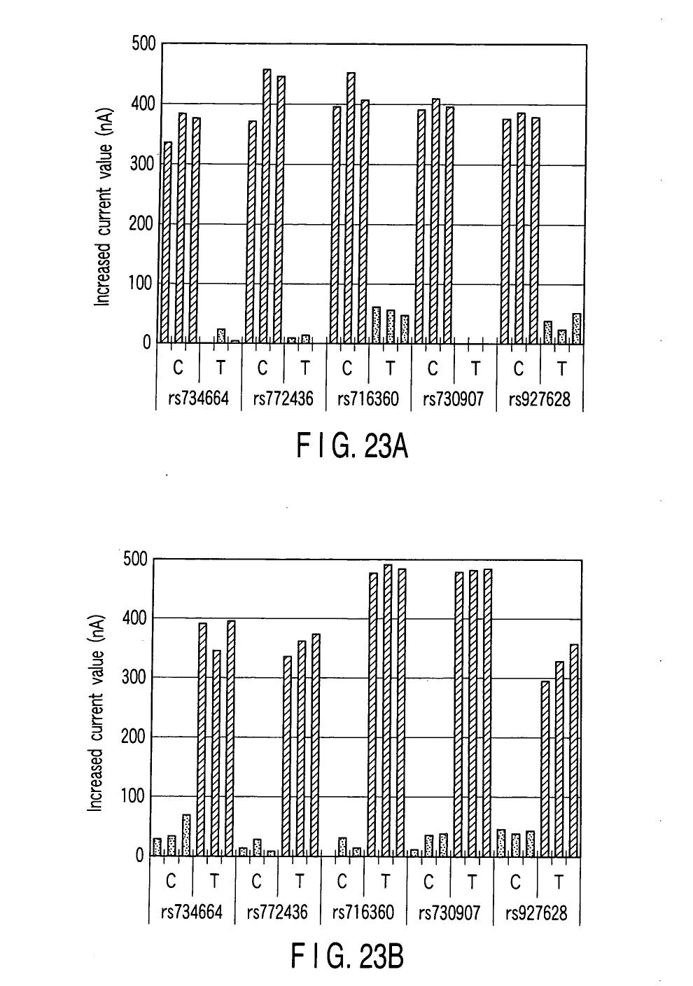

A peak current value obtained from each electrode is shown in FIG. 23. FIG. 23A is test result of a sample 1. A sample 1 clearly all showed a C/C homo type. Therefore, from values of Table 1, it becomes possible to determine that an individual having this genotype is present at one person per 696 persons.

On the other hand, a sample 2 shown in FIG. 23B all shows a T/T homo, and from values of Table 1, it becomes possible to determine one person per 5979 persons.

As described above, according to the present invention, a plurality of single nucleotide polymorphisms which are advantageous for individual discrimination can be selected, and the number of single nucleotide polymorphisms necessary for individual discrimination can be minimized. Thereby, a simple, rapid and economic individual discriminating method can be provided.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A subject individual discriminating method for determining consistency between a nucleotide sequence possessed by the subject individual and a nucleotide sequence possessed by a sample, the method comprising:
a step of selecting a plurality of single nucleotide polymorphisms from a group of single nucleotide polymorphisms present in a population to which the subject individual to be discriminated belongs; and
a step of determining genotypes in the selected plurality of single nucleotide polymorphisms in the nucleotide sequence possessed by the subject individual and the nucleotide sequence derived from a sample; and
a step of determining the consistency by comparing the genotypes,
wherein in the selecting step, single nucleotide polymorphism satisfying any one of the following conditions (i) to (iii) is selected:
(i) in case of a 2-nucleotide substitution type single nucleotide polymorphism, assuming that respective allele frequencies of two possible nucleotides are X and Y, X+Y=1, and Y≦X;
0.5≦X≦0.7,
(ii) in case of a 3-nucleotide substitution type single nucleotide polymorphism, assuming that respective allele frequencies of three possible nucleotides are X, Y and Z, X+Y+Z=1, and Z≦Y≦X;
1/3≦X and (1-X)/2≦Y and X+Y<1, and
(a) Y≦1/2·X and X<2/3, or
(b) 1/2·X<Y and XY<2/9, and
(iii) in case of a 4-nucleotide substitution type single nucleotide polymorphism, assuming that respective allele frequencies of four possible nucleotides are X, Y, Z and W, X+Y+Z+W=1, and W≦Z≦Y≦X;
1/4≦X and (1-X)/3≦Y and X+Y<1, and
(a) Y≦1/2·X and X<2/3, or
(b) 1/2·X<Y and XY<2/9.

2. The method according to claim 1, wherein the allele frequency in the condition (i) satisfies 0.55≦X<0.7.

3. The method according to claim 1, wherein the allele frequency in the condition (i) satisfies 0.6≦X<0.7.

4. The method according to claim 1, wherein the allele frequency in the condition (i) satisfies 0.65≦X<0.68.

5. The method according to claim 1, further comprising:
a step of determining reliability of result of the step of determining the consistency by calculating a probability that a combination of genotypes possessed by the subject individual is present in the population,
wherein the probability is calculated by multiplying genotype frequencies and taking a reciprocal,
and the genotype frequencies is calculated from allele frequencies regarding all of the selected single nucleotide polymorphisms of genotypes possessed by the subject individual.

6. An array in which nucleic acid probes are fixed on a substrate, for determining consistency between a nucleotide sequence possessed by an subject individual and a nucleotide sequence possessed by a sample, wherein
the nucleic acid probes have a sequence complementary with a target sequence containing single nucleotide polymorphism, and
the single nucleotide polymorphism is selected from a group of single nucleotide polymorphisms present in a population to which a subject individual to be discriminated belongs, and satisfies any one of the following conditions (i) to (iii):
(i) in case of a 2-nucleotide substitution type single nucleotide polymorphism, assuming that respective allele frequencies of two possible nucleotides are X and Y, X+Y=1, and Y≦X;
0.5≦X≦0.7,
(ii) in case of a 3-nucleotide substitution type single nucleotide polymorphism, assuming that respective allele frequencies of three possible nucleotides are X, Y and Z, X+Y+Z=1, and Z≦Y≦X;
1/3≦X and (1-X)/2≦Y and X+Y<1, and
(a) Y≦1/2·X and X<2/3, or
(b) 1/2·X<Y and XY<2/9, and
(iii) in case of a 4-nucleotide substitution type single nucleotide polymorphism, assuming that respective allele frequencies of four possible nucleotides are X, Y, Z and W, X+Y+Z+W=1, and W≦Z<Y≦X;
1/4≦x and (1-X)/3≦Y and X+Y<1, and
(a) Y≦1/2·X and X<2/3, or
(b) 1/2·X<Y and XY<2/9.

7. The array according to claim 6, wherein the allele frequency in the condition (i) satisfies 0.55≦X<0.7.

8. The array according to claim 6, wherein the allele frequency in the condition (i) satisfies 0.6≦X<0.7.

9. The array according to claim 6, wherein the allele frequency in the condition (i) satisfies 0.65≦X≦0.68.

10. An individual discriminating test apparatus, comprising:
an array according to claim 6;
a flow channel which is provided on a substrate of the array, and is provided along a direction of flow of a drug solution or the air;
working electrodes each of which is provided on the substrate at a plurality of numbers along the flow channel, and on which the probe is immobilized;
counter electrodes each of which is arranged on an internal circumferential surface of the flow channel corresponding to the working electrodes, and imparts an electric potential difference with the working electrodes;
reference electrodes each of which is arranged on an internal circumferential surface of the flow channel corresponding to the working electrodes and feed-backs a detected voltage to the working electrodes;
an inlet port which is opened in the flow channel, and flows a drug solution or the air into the flow channel from an upstream side of the flow channel;
an outlet port which is opened in the flow channel, and flows a drug solution or the air from the flow channel to a downstream side of the flow channel; and
an injection port which inject test solution into the flow channel.

11. An individual discriminating test system, comprising:
an individual discriminating test apparatus according to claim 10;
a supply system comprising a first piping which is communicated with the inlet port and supplies a drug solution or the air into the flow channel via the inlet port, and a first valve which controls a flow rate of a drug solution or the air of the first piping; and a discharge system comprising a second piping which is communicated with the outlet port and discharges a drug solution or the air from the flow channel via the outlet port, a second valve which controls a flow rate of a drug solution or the air of the second piping, and a pump which is provided in a second piping and draws up a drug solution or the air from the flow channel;
a measuring system comprising a voltage applying unit which imparts an electric potential difference between the working electrodes and the counter electrodes;
a temperature control system which controls a temperature of the array;
a control mechanism which controls the first valve of the supply system, the second valve and the pump of the discharge system, the voltage applying unit of the measurement system, and the temperature controlling system, the control mechanism detecting an electrochemical reaction signal from the working electrodes or the counter electrodes and storing the electrochemical reaction signal as measurement data; and
a computer which imparts a control condition parameter to the control mechanism to control the control mechanism, and at the same time, executes a processing of analyzing a nucleotide sequence based on the measurement data, and determines consistency between a nucleotide sequence possessed by an individual and nucleotide sequence possessed by a sample.
